(19) United States
(12) Patent Application Publication
(10) Pub. No.: US 2026/0191824 A1
(43) Pub. Date: Jul. 9, 2026

(54) TREATMENT OF OPTIC NEUROPATHIES

(71) Applicant: Oculis Operations Sàrl, Lausanne (CH)

(72) Inventors: Riad Sherif, Lausanne (CH); Pablo Villoslada Diaz, Palo Alto, CA (US)

(73) Assignee: Oculis Operations Sàrl, Lausanne (CH)

(21) Appl. No.: 19/438,847

(22) Filed: Jan. 2, 2026

Related U.S. Application Data
(60) Provisional application No. 63/741,709, filed on Jan. 3, 2025.

(30) Foreign Application Priority Data
Jan. 3, 2025 (EP) 25150260.5

(51) Int. Cl. A61K 31/4015; A61K 9/00; A61P 27/02

(52) U.S. Cl. CPC A61K 31/4015; A61K 9/0019; A61P 27/02

(57) ABSTRACT

A method of treating optic neuropathy that provides surprising efficacy while minimizing toxicity using a specific administration regimen with an effective amount of a compound of Formula (I):

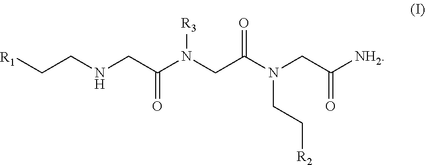

### CROSS-REFERENCE TO RELATED APPLICATIONS

This application claims the benefit of U.S. Provisional Application No. 63/741,709, filed Jan. 3, 2025, and European Patent Application No. 25150260.5, filed in the European Patent Office on Jan. 3, 2025. The entirety of each of these applications is incorporated herein for all purposes.

### FIELD OF THE INVENTION

This invention is in the field of treatments for optic neuropathies. The invention provides surprising efficacy while minimizing toxicity, in particular in subjects having acute optic neuropathy with visual impairments. The invention also includes specific dosing regimen, and patient selection criteria for the treatment, and resulting beneficial effects.

### BACKGROUND

Optic neuropathies are diseases that occur when the nerve fibers that transfer visual information from the eye to the brain are damaged. Optic neuropathy can lead to pain, loss of vision and blindness.

Optic neuropathies include, but are not limited to, acute optic neuritis (AON), anterior ischemic optic neuropathy, toxic or traumatic tumor-related optic neuropathies, genetic (Leber and Dominant Optic Atrophy) and glaucoma.

Inflammatory neurological conditions that result in the destruction or degeneration of neurons, axons or myelin and thus affect the central nervous system can result in optic neuritis, which is a form of optic neuropathy that is linked to an underlying inflammation-related disease such as multiple sclerosis (MS), Neuromyelitis Optica (NMO), MOG Associated Disease (MOGAD), transverse myelitis, acute disseminated encephalomyelitis, progressive multifocal leukoencephalopathy, central pontine myelinolysis, lupus or an infection. Conditions that cause inflammation can lead to swelling of the optic nerve, which can damage the myelin, or protective sheath, that surrounds the nerve, as well as the nerve itself.

Acute optic neuritis (AON) is an inflammation of the optic nerve that can cause demyelination and axonal loss of the optic nerve and the death of retinal ganglion cells. A variety of infectious diseases, immune disorders, demyelinating disorders, non-inflammatory systemic diseases or trauma can induce AON. AON is commonly associated with multiple sclerosis. AON is the first symptom of multiple sclerosis in 25% of cases and occurs during the course of the disease in 70% of cases (Toosy, A. T., Mason, D. F., & Miller, D. H. (2014). Optic neuritis. The Lancet Neurology, 13 (1), 83-99).

AON can also be observed in patients with no identified neurological or systemic disorders. In this case, AON is described as “idiopathic” and it is assumed that the pathogenesis is demyelination (Wikström, J. (1975). The epidemiology of optic neuritis in described in Finland, Acta Neurologica Scandinavica, 52 (3), 196-206).

At the onset of optic neuropathy that includes demyelination, such as AON, patients often suffer from ocular pain that increases with eye movement, and is associated with a variety of visual impairments. Deterioration of visual acuity, color vision or positive symptoms like flashes of light are common. The loss of vision varies among patients, ranging from mild blurring to loss of perception of light.

In the majority of cases of optic neuropathy such as AON, the condition tends to worsen over the first several days after the appearance of the symptoms and then starts to improve within the first two weeks. The recovery of symptoms can continue for up to a year after onset.

It has been observed that when inflammation recedes, remyelination occurs but it is usually incomplete. After a few weeks post onset, vision recovers but only partially, due to a persistent demyelination, axonal loss and neuronal death. Because the myelin sheath normally protects the axon, neurons with demyelinated segments post inflammation become fragile and prone to cellular death. The thinning of the Retinal Nerve Fiber Layer (RNFL) of the retina (constituted of unmyelinated axons originating from the retinal ganglion cell bodies) indicates significant axonal loss after AON. The RNFL is the thinnest between 3-6 months after an AON event, stabilizing after 6 months. Thinning of RNFL and thinning of Ganglion Cell Inner Plexiform Layer (GCIPL), the layer of ganglion cell bodies, correlates with diminished scores of visual acuity and diminished scores of visual field sensitivity.

Even where high contrast vision acuity eventually returns to near normal, patients often report that their vision is not fully recovered. There is a clinically meaningful reduction in vision-related quality of life (as shown by the National Eye Institute Visual Function Questionnaire (Guy, J., Fitzsimmons, J., Ellis, E. A., Beck, B., & Mancuso, A. (1992)). Intraorbital optic nerve and experimental optic neuritis: correlation of fat suppression magnetic resonance imaging and electron microscopy. Ophthalmology, 99 (5), 720-725). Persistent impairment of low contrast letter acuity can be experienced, even when they have recovered up to 20/40 high contrast visual acuity or above. These impairments are correlated with decreased Retinal Nerve Fiber Layer (RNFL) and GCIPL thickness Id.

Inflammation that leads to the demyelination of the optic nerve, and then to the loss of nerve fibers, thus can produce long-term, irreversible visual impairments.

To date, there is no approved treatment for demyelinating optic neuropathy including acute optic neuritis (AON), either idiopathic or associated with multiple sclerosis or other inflammatory-causing condition.

Typically, patients who experience inflammation-related demyelination receive high dose steroids intravenously for a few days to accelerate the resolution to a residual dysfunction. As corticosteroids (e.g. methylprednisolone) shorten the attack and hasten recovery from acute visual symptoms, they have become the standard of care. However, this treatment has not been shown to prevent axonal loss nor to improve the final visual prognosis.

Therefore, there is an urgent need for a treatment of inflammatory demyelinating optic neuropathy that improves final visual prognosis and/or ganglion/retinal nerve fiber integrity.

### SUMMARY

The present disclosure provides a method for the treatment of a host such as a human with an optic neuropathy based on administering a low dose effective amount of a compound of Formula (I) below, e.g. OCS-05 (Formula II), which is a serum-glucocorticoid (SGK2) agonist and FOXO3 agonist, or a pharmaceutically acceptable salt thereof, in a manner that improves or restores vision and/or ganglion/retinal nerve fiber integrity. Hence, the present disclosure more specifically provides methods for treating visual impairments in a human subject, said method comprising administering an effective amount of a compound of Formula (I) below, e.g. OCS-05 (Formula II). This invention is based on human clinical trial data, not on an animal model, and therefore is precise for humans.

In a specific embodiment, the methods of the present disclosure provide superior dosage regime consisting of a single regimen of a once daily low dose efficacious parenteral administration, for example, intravenous, of 2-6 mg/kg (for example 2-4, such as 3 mg/kg) of the selected compound of Formula (I), for 4-6 continuous days (for example, 5 continuous days). In an optimal embodiment, the treatment is complete at the end of this 4-6 day regimen. In an alternative embodiment, if the day or days within the 4-6 continuous days requires that the day or days fall on a weekend or holiday, a variation can be allowed as long as the total days of the single treatment cycle does not exceed ten days.

It has been also been found that this low dose of a compound of Formula I, for example OCS-05 is appropriate in humans to treat optic neuropathy based on actual human clinical trials, This is lower than expected based on animal models.

This superior treatment should preferably be initiated within 12 days of onset of symptoms, such as loss in Low Contrast Visual Acuity after onset of acute optic neuritis. The described treatment can optionally be administered on top of standard of care. In certain embodiments, the patient may receive a steroid injection, which in a nonlimiting embodiment is administered on the days of treatment. In a certain embodiment, the method includes a single treatment regimen which does not have to be repeated after completion, and thus is not a “discontinuous treatment regime” that requires multiple rounds of treatment with interspersed periods of no treatment. This single regimen provides convenience for the patient and results in effective treatment of optic neuritis without undue toxicity.

In a certain embodiment, the human patient has demyelinating unilateral or bilateral acute optic neuritis. In other embodiments, the human patient has anterior ischemic optic neuropathy, Leber disease, dominant optic atrophy, glaucoma, and toxic or traumatic tumor-related optic neuropathy. In some embodiments, the human patient has an underlying inflammation-related disease such as multiple sclerosis (MS), Neuromyelitis Optica (NMO), MOG associated disease (MOGAD), lupus or an inflammation-causing infection.

In an alternative embodiment, the dosage regimen can be provided as a subcutaneous injection. The dosage can be provided as either a standard mg per injection, or can be measured in mg/kg. If the latter, 1-3 mg/kg (including 1, 2, or 3 mg/kg, for example 2 mg/kg) once a day for 4-6 days (for example 5 days) may be typical.

Other routes of administration include intravitreal injection, or an ocular topical formulation, which provides, for example, 200-400 μg/mL for topical administration, and 1 to 10 μg/eye for intravitreal injection, for example about 5 μg/eye of the selected compound of Formula (I), for example, Formula (II), (III) or (IV). Alternative forms of administration include intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, and tear duct injections, or through a mucus, mucin, or a mucosal barrier, in an immediate or controlled release fashion.

Formula (I) is:

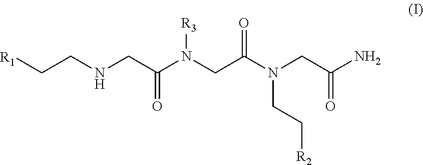

wherein:
R₁ is phenyl substituted with halogen or trifluoromethyl, and further optionally substituted with one or two substituents independently selected from the group consisting of halogen, (C₁₋₆) alkyl, (C₁₋₆)alkoxy, and halo(C₁₋₆)alkyl; or
R₁ is pyrrolidin-1-yl;
R₂ is

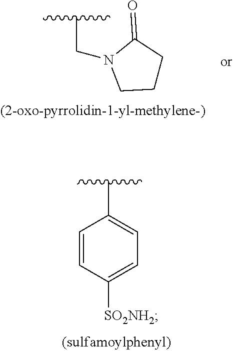 [SMILES: *CN1CCCC1=O ; *c1ccc(S(N)(=O)=O)cc1]

and
R₃ is selected from methyl, ethyl, propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl.

In another embodiment, the compound of formula (I) is selected from the group consisting of formula (II) (OCS-05), formula (III) or formula (IV), or its pharmaceutically acceptable salt.

In specific embodiments, a compound of the Formula II, III or IV, or its pharmaceutically acceptable salt is provided in the superior dosage formulation.

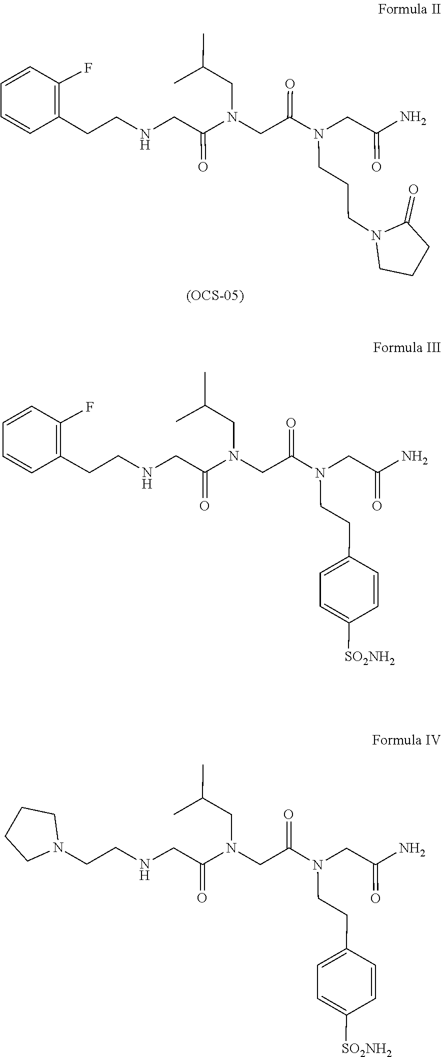 [SMILES: CC(C)CN(CC(=O)N(CCCN1CCCC1=O)CC(N)=O)C(=O)CNCCc1ccccc1F ; CC(C)CN(CC(=O)N(CCc1ccc(S(N)(=O)=O)cc1)CC(N)=O)C(=O)CNCCc1ccccc1F ; CC(C)CN(CC(=O)N(CCc1ccc(S(N)(=O)=O)cc1)CC(N)=O)C(=O)CNCCN1CCCC1]

The peptidomimetic structures of Formulas I, II (OCS-05), III and IV were first disclosed in WO2012/028959 to Villoslada and Messeguer as agonists of neurotropin receptors for treatment of a very broad range of neurological or psychiatric disorders or aging (see Paragraphs 0121-0122). No particular dosage regimen was disclosed, nor a specific effect of any of those compounds in improving visual impairments such as Low Contrast Visual Acuity, after onset of acute optic neuritis. The '959 application in Paragraph 0110 defined an effective amount very broadly in milligrams ranging from 0.01 mg to 50 grams per day, and narrowed to 10 mg to 500 mg per day. Further, while it was mentioned that the pharmaceutical composition can be administered in any means that achieves the intended purpose (Paragraph 0130), the route singled out was oral, which is consistent with using milligrams or grams as the dosage amount (instead of mg/kg which is the standard intravenous or subcutaneous route).

In 2021, WO 2021/084013 was published, naming Bionure Farma, S. L. as the assignee and Villoslada as the inventor, which referenced the same class of peptidomimetics. The '013 publication highlighted, among other things, a specific discontinuous dosage regimen that consisted of (a) a first time period from 1 to 7 days, wherein the compound is administered once or several times to a subject in need thereof, followed by (b) a second time period equal to or longer than 13 days, wherein the compound is not administered, and wherein the second time period takes place after the first time period and before the next administration of the compound. Therefore, the '013 publication highlighted that Formula II (OCS-01) or more generally a compound of Formula I shows efficacy well past the time of administration. In one embodiment, it described a first dosage over 1-7 days, a rest period of at least 13 days, followed by a second treatment period (see for example the Abstract and claim 1 as published). This was referred to in the '013 publication as a “discontinuous treatment regimen” (see page 1, lines 8-10). Interestingly, the '013 application defines the dosage of the compound in terms of a conversion from a mouse dosage, wherein the mouse dosage is 0.5-200 mg/kg, which may equate to 0.04 mg/kg to 16.26 mg/kg in humans (see '013 publication, page 20). The present research and discovery based on human clinical trials has found that there is a small window of human dosage administration that is both efficacious and sufficiently non-toxic to humans, and that range is about 1-5 mg/kg (such as 1, 2, 3, 4, or 5 mg/kg) and typically 3 mg/kg.

In sharp contrast, it has been discovered that a single treatment period of a once daily low dose systemic, for example intravenous, administration of 2, 3, 4, or 5 mg/kg (usually 3-4 mg/kg) of the selected compound of Formula (I) (for example 3 mg/kg), for 4-6 continuous days (for example, 5 continuous days) satisfies the criteria of good efficacy and low toxicity. Typically, the treatment can be complete at the end of this single 4-6 day regimen, which can be stretched to 4-6 dosages once a day over ten days at the most if it is inconvenient to get the intravenous infusions for 4-6 days in a row.

It has been discovered that, for example, intravenous administration of about 2, 3, 4, or 5 mg/kg, for example 3-4 mg/kg, of the selected compound of Formula (I), and in particular Formula II (OCS-05) for 4-6 continuous days (for example, 5 continuous days), provides at least the following surprising combined benefits (using OCS-05 at 3 mg/kg/day). This has been confirmed in a Phase 2 human clinical trial (ACUITY).
(i) A statistically significant difference in GCIPL thickness as a surrogate of retinal ganglion cell preservation of up to 43% extended to at least month 3, 4 or 5 from administration (Example 2, FIGS. 1-2).
(ii) A statistically significant difference in RNFL thickness as a surrogate of axon preservation of up to 28% extended to at least month 3, 4 or 5 from administration (Example 2, FIGS. 3-4).
(iii) A statistically significant improvement in low contrast visual acuity (LCVA) extended to at least month 3 from administration (Example 3, FIGS. 5-6).
(iv) No drug-related serious adverse events or adverse events leading to drug withdrawal (i.e., low toxicity).
(v) Lower incidence of adverse events related to multiple sclerosis relapses or worsening of CNS inflammatory disorders.
(vi) No difference in the percentage of patients with abnormal electrocardiogram (ECG) events between dosed patients and placebo patients.

In general, a compound of Formula I (e.g. Formula II in human clinical trials) shows a favorable safety profile, improvement in visual function and preservation of retinal ganglion cells and optic nerve structure. These results demonstrate superior and excellent performance.

In an earlier Phase I safety-only human clinical trial using standard single and multiple dose ascending studies (SAD and MAD) with representative Formula I compound OCS-05, it was established that IV administration for up to 5 days showed a safe profile and linear pharmacokinetics. Efficacy was not established or evaluated. Villoslada, et. al., “A Phase 1 randomized study on the safety and pharmacokinetics of OCS-05, a neuroprotective disease modifying treatment for Acute Optic Neuritis and Multiple Sclerosis; Scientific Reports, (2023) 13:5099.

In the cell, the compounds of Formula I, for example, Formula II (OCS-05), bind selectively to a small group of kinases of the Insulin Growth Factor 1 (IGF-1) pathway, namely Serum Glucocorticoid Kinase 2 (SGK-2). OCS-05 promotes the translocation out of the nucleus of the transcription factor FOXO-3, highly expressed in the central nervous system. FOXO-3's translocation inhibits the expression of pro-apoptotic genes and induces the expression of anti-apoptotic genes, antioxidant enzymes, and differentiation pathways.

Nonclinical in-vitro and in-vivo studies showed that OCS-05 has neuroprotective and remyelinating properties (Villoslada et al., Neurotherapeutics, doi.org/10.1007/s13311-019-00717-4). A full program of safety and toxicology nonclinical studies was conducted to characterize the safety profile of OCS-05, (NCT04762017, Villoslada et al., 2023 Nature, Scientific Reports, 13:5099).

The phase 1 study investigated the safety and tolerability of single and multiple doses of intravenous OCS-05 in healthy volunteers and characterized its pharmacokinetic profile.

In certain embodiments, the present disclosure also includes patient selection inclusion and exclusion for the disclosed treatment regimen.

Patient inclusion criteria, for example, includes:
(1a) Diagnosed with an optic neuropathy that is demyelinating, which may in an embodiment be acute optic neuritis; or
(2a) Onset of visual loss symptoms within the last 12 days before treatment begins. Patient exclusion criteria, for example, can include:
(1b) Optic neuropathy of non-demyelinating origin; or
(2b) Known neuromyelitis optica with autoantibodies against aquaporin-4 (AQP4-Abs).

In summary, the disclosure provides at least the following methods:
(i) A method for treating visual impairments in a human subject having an optic neuropathy, said method comprising parenterally administering an effective amount of a compound of Formula I or a pharmaceutically acceptable salt thereof,
(ii) a method for the treatment of optic neuropathy, comprising parenterally administering to a human in need thereof an effective amount of a compound of Formula I or a pharmaceutically acceptable salt thereof, with a single treatment regimen of a once daily administration of 2-6 mg/kg (for example about 2, 3, 4, 5, or 6 mg/kg) for 4, 5, or 6 continuous days;
(iii) a method for the treatment of optic neuropathy, comprising parenterally administering to a human in need thereof an effective amount of a compound of Formula I or a pharmaceutically acceptable salt thereof, with a single regimen of 2-6 mg/kg (for example about 2, 3, 4, 5, or 6 mg/kg) for 4, 5, or 6 daily administrations spread over not longer than ten days;
(iv) the method of (i), (ii) or (iii) wherein the compound of Formula I or a pharmaceutically acceptable salt thereof is Formula II or a pharmaceutically acceptable salt thereof;
(v) the method of any one of (i)-(iv), wherein about 3 mg/kg of the compound is administered intravenously;
(vi) the method of any one of (i)-(v), wherein about 3 mg/kg of the compound is administered subcutaneously;
(vii) the method of any one of (i)-(iii), wherein between 1 and 10 μg/eye of the compound is administered intravitreally, for example about 1 μg/eye, 2 μg/eye, 3 μg/eye, 4 μg/eye, 5 μg/eye, 6 μg/eye, 7 μg/eye, 8 μg/eye, 9 μg/eye, or 10 μg/eye;
(viii) the method of any one of (i)-(iii), wherein 100-500 μg/ml of the compound is administered topically, typically one drop a day; for example about 100 μg/mL, 150 μg/mL, 200 μg/mL, 250 μg/mL, 300 μg/mL, 350 μg/mL, 400 μg/mL, 450 μg/mL, or 500 μg/mL;
(ix) the method of any one of (i)-(viii), wherein the compound is administered for 5 days;
(x) the method of (v) or (vi), wherein the about 3 mg/kg of the compound is administered for 5 days;
(xi) the method of (vii) or (viii), wherein the about 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg of the compound is administered for 5 days;
(xii) the method of any one of (i)-(xi), wherein treatment is initiated within 12 days of onset of symptoms;
(xiii) the method of any one of (i)-(xii), wherein the treatment is administered on top of standard of care;
(xiv) the method of (xiii), wherein the standard of care is steroid injection;
(xv) A compound of Formula I, or a pharmaceutically acceptable salt thereof, for use in a method for treating visual impairments in a human subject having an optic neuropathy, said method comprising parenterally administering an effective amount of a compound of Formula I or a pharmaceutically acceptable salt thereof,
(xvi) a compound of Formula I, or a pharmaceutically acceptable salt thereof, for use in a therapeutic amount in the treatment of optic neuropathy in a human in need thereof comprising parenterally administering the compound in a single treatment regimen of a once daily administration of 2-6 mg/kg (for example about 2, 3, 4, 5, or 6 mg/kg) for 4, 5, or 6 continuous days;
(xvii) a compound of Formula I, or a pharmaceutically acceptable salt thereof, for use in a therapeutic amount in the treatment of optic neuropathy in a human in need thereof comprising parenterally administering 2-6 mg/kg (for example about 2, 3, 4, 5, or 6 mg/kg) of the compound in a single regimen of 4, 5, or 6 daily administrations spread over not longer than ten days;
(xviii) the compound for use of (xv) or (xvii), wherein the compound of Formula I or a pharmaceutically acceptable salt thereof is Formula II or a pharmaceutically acceptable salt thereof;
(xix) the compound for use in any one of (xv)-(xvii), wherein about 3 mg/kg of the compound is administered intravenously;
(xx) the compound for use in any one of (xv)-(xvii), wherein about 3 mg/kg of the compound is administered subcutaneously;
(xxi) the compound for use in any one of (xv)-(xviii), wherein between 1 and 10 μg/eye of the compound is administered intravitreally, for example about 1 μg/eye, 2 μg/eye, 3 μg/eye, 4 μg/eye, 5 μg/eye, 6 μg/eye, 7 μg/eye, 8 μg/eye, 9 μg/eye, or 10 μg/eye;
(xxii) the compound for use in any one of (xv)-(xviii), wherein between 100 and 500 μg/mL of the compound is administered topically, for example about 100 μg/mL, 150 μg/mL, 200 g/mL, 250 μg/mL, 300 μg/mL, 350 μg/mL, 400 μg/mL, 450 μg/mL, or 500 μg/mL, typically one drop a day;
(xxiii) the compound for use any one of (xv)-(xii), wherein the compound is administered for 5 days;
(xxiv) the compound for use in any one of (xv)-(xxiii), wherein the about 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg of the compound is administered for 5 days;
(xxv) the compound for use in any one of (xv)-(xxiv), wherein treatment is initiated within 12 days of onset of symptoms;
(xxvi) the compound for use in any one of (xv)-(xxv), wherein the treatment is administered in combination with a steroid therapy; and
(xxvii) the compound for use of (xxvi), wherein the steroid therapy is a standard of care.

### BRIEF DESCRIPTION OF THE FIGURES

FIG. 1 shows the therapeutic effect of OCS-05 (Formula II) on ganglion cell and inner plexiform layer (GCIPL) thickness in the affected eye over time. Treatment with 3 mg/kg/d OCS-05 resulted in a 43% improvement in GCIPL thickness at three and six months after treatment. As described in Example 2, the GCIPL thicknesses plotted are the mean change from baseline measurements.

FIG. 2 shows the therapeutic effect of OCS-05 on ganglion cell and inner plexiform layer (GCIPL) thickness in the affected eye over time. Treatment with either 2 mg/kg/d or 3 mg/kg/d OCS-05 resulted in statistically significant improvement in GCIPL thickness at three and six months after treatment. The p-values are shown in the plot. As described in Example 2, the GCIPL thicknesses plotted are the mean change from baseline measurements.

FIG. 3 shows the clinically meaningful therapeutic effect of OCS-05 on retinal nerve fiber layer (RNFL) thickness in the affected eye over time. Treatment with 3 mg/kg/d OCS-05 resulted in a 28% improvement in RNFL thickness at three months and a 30% improvement at six months after treatment. As described in Example 2, the RNFL thicknesses plotted are the mean change from baseline measurements.

FIG. 4 shows the statistically significant therapeutic effect of OCS-05 on retinal nerve fiber layer (RNFL) thickness in the affected eye over time. Treatment with 3 mg/kg/d OCS-05 resulted in a statistically significant improvement in RNFL thickness at three months (p=0.045) and six months (p=0.033) after treatment. As described in Example 2, the RNFL thicknesses plotted are the mean change from baseline measurements.

FIG. 5 shows the statistically significant and clinically meaningful improvement in low contrast visual acuity in subjects in the OCS-05 3 mg/kg/d. At 3 months, the subjects in the treatment arm scored 18 letters (3 lines and a half) better than the placebo arm in the LCVA examination (p=0.004). At 6 months, the subjects in the treatment arm scored 15 letters better than the placebo arm in the LCVA examination (p=0.012). The protocol for the LCVA examination is described in Example 3.

FIG. 6 shows the statistically significant and clinically meaningful improvement in low contrast visual acuity in subjects in both treatment arms (OCS-05 3 mg/kg/d and 2 mg/kg/d). At 3 months, the subjects in the 3 mg/kg/d treatment arm scored 18 letters better than the placebo arm in the LCVA examination (p=0.004). The subjects in the 2 mg/kg/d treatment arm scored 15 letters (3 lines) better than placebo in the LCVA examination (p=0.106). At 6 months, the subjects in the 3 mg/kg/d treatment arm scored 15 letters better than the placebo arm in the LCVA examination (p=0.012). At 6 months, the subjects in the 2 mg/kg/d treatment arm scored 8 letters better than the placebo arm in the LCVA examination (p=0.345). The protocol for the LCVA examination is described in Example 3.

FIG. 7 shows the change from baseline in plasma neurofilaments (NfL) for OCS-05 groups (2 and 3 mg/kg/d and both doses pooled) and placebo. (abbreviations: D, day; LS, least-squares; M, month; NfL, neurofilament light chain).

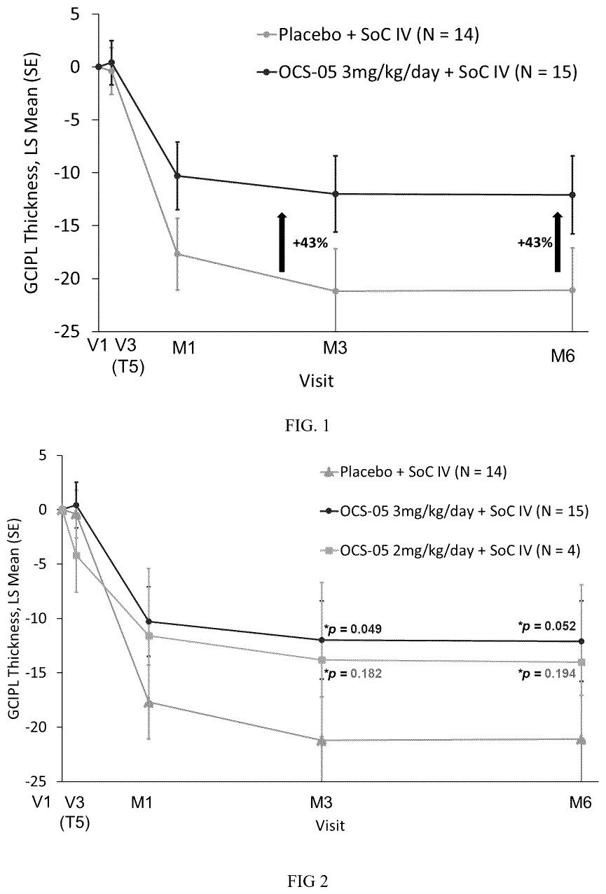

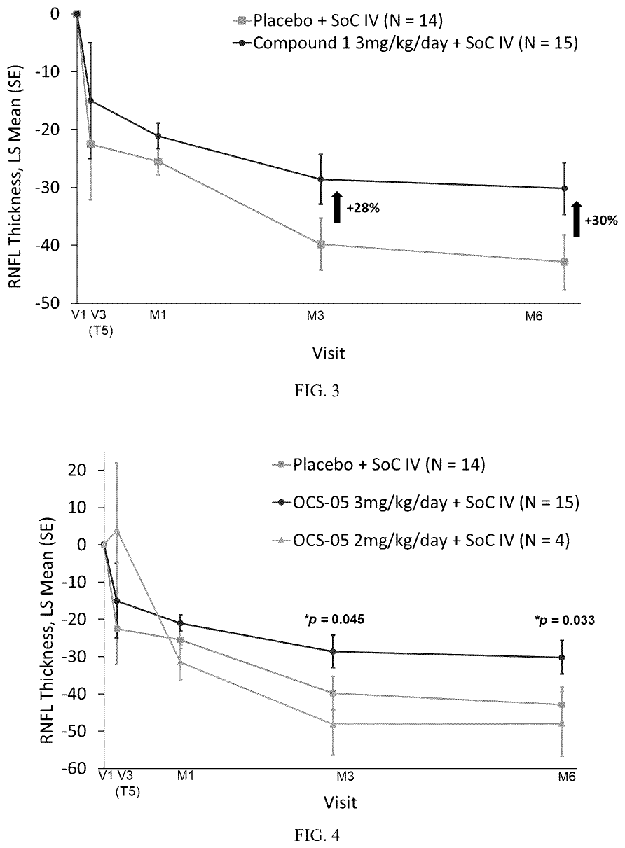

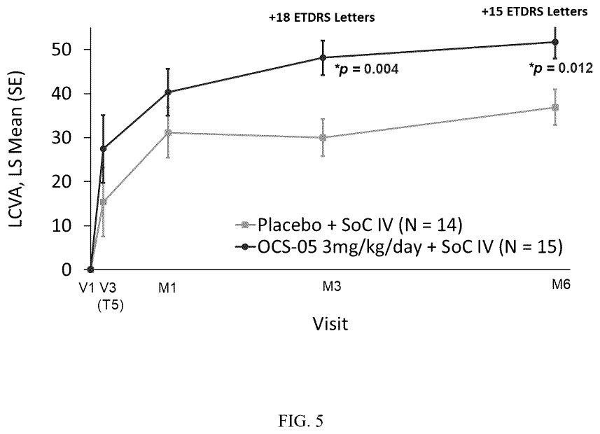

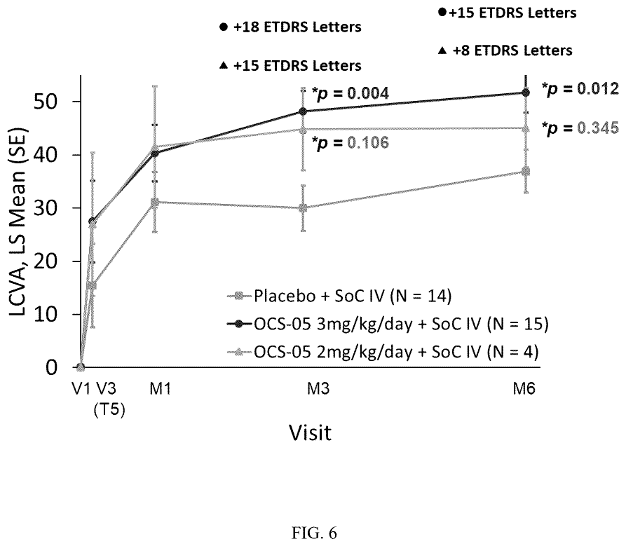

### DETAILED DESCRIPTION

The present disclosure provides a method for the treatment of a human with an optic neuropathy based on administering an effective low dose amount of a compound of Formula (I) below, which is a serum-glucocorticoid (SGK2) and FOXO3 agonist, or a pharmaceutically acceptable salt thereof, in a manner that improves or restores vision and/or final vision prognosis, and/or ganglion/retinal nerve fiber integrity.

In a specific embodiment, the method consists of a single regimen of a once daily parenteral (for example intravenous) administration of 2-6 mg/kg (for example about 3 mg/kg) of the selected compound of Formula (I), e.g., OCS-05 (Formula II), for 4-6 continuous days (for example, 5 continuous days). In an optimal embodiment, the treatment is complete at the end of this 4-6 day regimen. In an alternative embodiment, if the day or days within the 4-6 continuous days requires that the day or days fall on a weekend or holiday, a variation can be allowed as long as the total days of the single treatment cycle does not exceed ten days.

In a specific embodiment, this method comprises the single treatment regimen which does not have to be repeated after completion, and thus is not a “discontinuous treatment regime” that requires multiple rounds of treatment with interspersed periods of no treatment. This single regimen provides convenience for the patient and results in effective treatment of the optic neuritis without undue toxicity.

In a specific embodiment, the treatment should preferably be initiated within 12 days of onset of symptoms. The described treatment can optionally be administered on top of steroid injection, for example, the current standard of care. Standard of care can be, for example, one dose of intravenous steroid injection per day on the days of treatment.

In a certain embodiment, the human patient has demyelinating unilateral or bilateral acute optic neuritis. In other embodiments, the human patient has anterior ischemic optic neuropathy (including Non-arteritic Anterior Ischemic Optic Neuropathy (NAION)), Leber disease, dominant optic atrophy, glaucoma, and toxic or traumatic tumor-related optic neuropathy. In some embodiments, the human patient has an underlying inflammation-related disease such as multiple sclerosis (MS), Neuromyelitis Optica (NMO), MOG Associated Disease (MOGAD), lupus or an inflammation-causing infection.

In an illustrative embodiment, a dosage regimen can be provided as a subcutaneous injection of 1-3 mg/kg (for example 2 mg/kg) once a day for 4-6 days (for example 5 days). In certain embodiments, the dosage regimen can be provided as a subcutaneous injection of 50-300 mg (for example 140 mg for a 70 kg patient) once a day for 4-6 days (for example 5 days). Other routes of administration include intravitreal injection (comprising, for example injecting about 1 μg/eye, 2 μg/eye, 3 μg/eye, 4 μg/eye, 5 μg/eye, 6 μg/eye, 7 μg/eye, 8 μg/eye, 9 μg/eye, or 10 μg/eye of a compound of Formula (I)), or an ocular topical formulation in a standardized topical dose, which provides 100 and 500 μg of the selected compound of Formula (I), for example, Formula (II), (III) or (IV), per mL of the ocular topical formulation. Alternative forms of administration include intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, and tear duct injections, or through a mucus, mucin, or a mucosal barrier, in an immediate or controlled release fashion.

Formula (I) is:

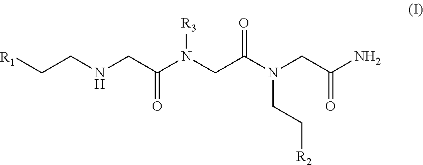

wherein:
R₁ is phenyl substituted with halogen or trifluoromethyl, and further optionally substituted with one or two substituents selected from the group consisting of halogen, (C₁₋₆) alkyl, (C₁₋₆) alkoxy, and halo(C₁₋₆)alkyl; or
R₁ is pyrrolidin-1-yl;
R₂ is

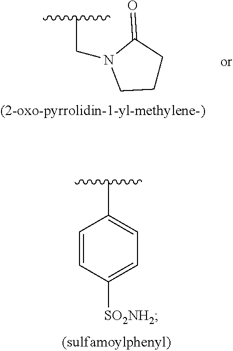 [SMILES: *CN1CCCC1=O ; *c1ccc(S(N)(=O)=O)cc1]

and
R₃ is selected from ethyl, propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl.

### Definitions

As used herein, the term “treating” includes reversing, alleviating or inhibiting the progression of optic neuropathy, including any of the types described herein, such as acute optic neuritis.

The term “host” or “patient” as used herein typically refers to a human. In other embodiments, the term host or patient can refer to a mammal that suffers from an optic neuropathy such as a dog, cat, horse, or bovine.

As used herein, the term “about” refers to the stated value plus or minus 10% of the stated value. For example, about 100 includes all values from 90 to 110.

As used herein, the term (C₁₋ₙ)alkyl refers to a saturated branched or linear hydrocarbon chain which contains from 1 to n carbon atoms and only single bonds. Non limiting examples of (C₁₋ₙ)alkyl include methyl, ethyl, 1-propyl, 2-propyl, 1-butyl, 2-methyl-1-propyl, and the like.

The term (C₁₋ₙ)alkoxy refers to a (C₁₋ₙ)alkyl as previously defined which is linked to the rest of a molecule or to another group through an oxygen atom. Non limiting examples of (C₁₋ₙ)alkoxy include methoxy, ethoxy, n-propoxy, isopropoxy, and the like.

The term halo(C₁₋ₙ)alkyl refers to a (C₁₋ₙ)alkyl as previously defined, where some or all of the hydrogen atoms are replaced by fluorine, chlorine, bromine and/or iodine. Non limiting examples of halo(C₁₋ₙ)alkyl include chloromethyl, difluoromethyl, trifluoromethyl, 2-fluoroethyl, 2,2-difluoroethyl, 2,2,2-trifluoroethyl and the like.

A halogen substituent means fluoro, chloro, bromo or iodo.

The expression “pharmaceutically excipients or carriers” refers to pharmaceutically or veterinary acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio.

The expression “effective amount” as used herein, refers to the amount of a compound that, when administered, is sufficient to prevent development of, or alleviate to some extent, one or more of the symptoms of the disease which is addressed. In particular, in reference to treating optic neuropathies in the context of the disclosed methods, the term “effective amount” may in some embodiments refer to an amount of a compound that, when administered is sufficient to alleviate visual impairments after onset of optic neuropathy, such as optic neuritis, for example but not limited to loss of Low Contrast Visual Acuity, in particular loss of visual impairments at 3 months, or at 6 months after onset of symptoms.

Some of the compounds of Formula (I) may have one or more chiral centers that can give rise to one or more stereoisomers. The term stereoisomer includes mirror image isomers (enantiomers), mixtures of mirror image isomers (racemates, racemic mixtures), geometric (cis/trans or syn/anti or E/Z) isomers, and isomers of compounds with more than one chiral center that are not mirror images of one another (diastereoisomers). The present disclosure includes the use independently of any of these stereoisomers in isolated or mixed form.

### The Compounds for Use in the Disclosed Methods

The present disclosure relates to methods of use of SGK2 agonist compounds, more specifically SGK2 and FOXO3 agonist, for treating acute neuropathies, for example acute optic neuritis, in particular for treating visual impairments, said method comprising administering to said subject an effective amount of an SGK2 agonist.

As used herein, the term “SGK2 agonist” refers to a compound that selectively enhances the activity of the Serum/Glucocorticoid-Regulated Kinase 2 (SGK2). SGK2 is a serine/threonine protein kinase involved in various cellular processes, including cell survival, proliferation, and ion transport. SGK2 agonists work by binding to the kinase domain of SGK2, promoting its active conformation, and increasing the phosphorylation of its substrates. This activation can influence multiple signaling pathways, depending on the specific targets involved. Examples of SGK2 agonist compounds are disclosed in WO2012/028959.

As used herein, the term “FOXO3 agonist” refers to a compound that promotes the translocation out of the nucleus of the transcription factor FOXO3, highly expressed in the central nervous system. FOXO3's translocation inhibits the expression of pro-apoptotic genes and induces the expression of anti-apoptotic genes, antioxidant enzymes, and differentiation pathways.

In an embodiment, an SGK2 and FOXO3 agonist for use in the methods of the present disclosure is a compound of the following formula (I), or pharmaceutically acceptable salts thereof:

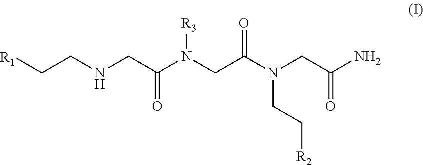

wherein:
R₁ is phenyl substituted with halogen or trifluoromethyl, and further optionally substituted with one or two substituents selected from the group consisting of halogen, (C₁₋₆) alkyl, (C₁₋₆) alkoxy, and halo(C₁₋₆)alkyl; or
R₁ is pyrrolidin-1-yl;
R₂ is

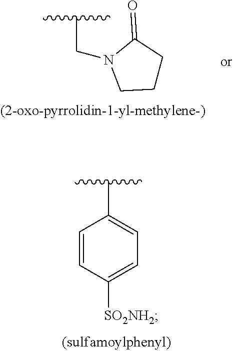 [SMILES: *CN1CCCC1=O ; *c1ccc(S(N)(=O)=O)cc1]

and
R₃ is selected from methyl, ethyl, propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl.

In one embodiment, in the compound of formula (I), R₁ is fluorophenyl, more particularly 2-fluorophenyl, 3-fluorophenyl or 4-fluorophenyl, and can be more particularly, 2-fluorophenyl.

In another embodiment, in the compound of formula (I), R₁ is fluorophenyl which is further substituted with one or two substituents selected from the group consisting of halogen, (C₁₋₆)alkyl, (C₁₋₆)alkoxy, and halo(C₁₋₆)alkyl; particularly R₁ is fluorophenyl which is further substituted with one or two substituents selected from the group consisting of halogen, (C₁₋₄)alkyl, (C₁₋₄)alkoxy, and halo(C₁₋₄)alkyl; even more particularly R₁ is fluorophenyl which is further substituted with one or two substituents selected from the group consisting of halogen, methyl, ethyl, propyl, isopropyl, methoxy, ethoxy, fluoromethyl, and trifluoromethyl.

In another embodiment, in the compound of formula (I), R₁ is chlorophenyl, more particularly 2-chlorophenyl, 3-chlorophenyl or 4-chlorophenyl, and even more particularly R₁ is 2-chlorophenyl.

In another embodiment, in the compound of formula (I), R₁ is chlorophenyl which is further substituted with one or two substituents selected from the group consisting of halogen, (C₁₋₆)alkyl, (C₁₋₆)alkoxy, and halo(C₁₋₆)alkyl; particularly R₁ is fluorophenyl which is further substituted with one or two substituents selected from the group consisting of halogen, (C₁₋₄)alkyl, (C₁₋₄)alkoxy, and halo(C₁₋₄)alkyl; even more particularly R₁ is fluorophenyl which is further substituted with one or two substituents selected from the group consisting of halogen, methyl, ethyl, propyl, isopropyl, methoxy, ethoxy, fluoromethyl, and trifluoromethyl.

In another embodiment, in the compound of formula (I), R₁ is bromophenyl, more particularly 2-bromophenyl, 3-bromophenyl or 4-bromophenyl, and even more particularly 2-bromophenyl.

In another embodiment, in the compound of formula (I), R₁ is bromophenyl which is further substituted with one or two substituents selected from the group consisting of halogen, (C₁₋₆)alkyl, (C₁₋₆)alkoxy, and halo(C₁₋₆)alkyl; particularly R₁ is bromophenyl which is further substituted with one or two substituents selected from the group consisting of halogen, (C₁₋₄)alkyl, (C₁₋₄)alkoxy, and halo(C₁₋₄)alkyl; even more particularly R₁ is bromophenyl which is further substituted with one or two substituents selected from the group consisting of halogen, methyl, ethyl, propyl, isopropyl, methoxy, ethoxy, fluoromethyl, and trifluoromethyl.

In another embodiment, in the compound of formula (I), R₁ is iodophenyl, more particularly 2-iodophenyl, 3-iodophenyl or 4-iodophenyl, and even more particularly 2-iodophenyl.

In another embodiment, in the compound of formula (I), R₁ is iodophenyl which is further substituted with one or two substituents selected from the group consisting of halogen, (C₁₋₆)alkyl, (C₁₋₆)alkoxy, and halo(C₁₋₆)alkyl; particularly R₁ is iodophenyl which is further substituted with one or two substituents selected from the group consisting of halogen, (C₁₋₄)alkyl, (C₁₋₄)alkoxy, and halo(C₁₋₄)alkyl; even more particularly R₁ is iodophenyl which is further substituted with one or two substituents selected from the group consisting of halogen, methyl, ethyl, propyl, isopropyl, methoxy, ethoxy, fluoromethyl, and trifluoromethyl.

In another embodiment, in the compound of formula (I), R₁ is trifluoromethylphenyl, more particularly 2-trifluoromethylphenyl, 3-trifluoromethylphenyl or 4-trifluoromethylphenyl, and even more particularly 2-trifluoromethylphenyl.

In another embodiment, in the compound of formula (I), R₁ is trifluoromethylphenyl which is further substituted with one or two substituents selected from the group consisting of halogen, (C₁₋₆)alkyl, (C₁₋₆)alkoxy, and halo(C₁₋₆)alkyl; particularly R₁ is trifluoromethylphenyl which is further substituted with one or two substituents selected from the group consisting of halogen, (C₁₋₄)alkyl, (C₁₋₄)alkoxy, and halo(C₁₋₄)alkyl; even more particularly R₁ is trifluoromethylphenyl which is further substituted with one or two substituents selected from the group consisting of halogen, methyl, ethyl, propyl, isopropyl, methoxy, ethoxy, fluoromethyl, and trifluoromethyl.

In another embodiment, in the compound of formula (I), R₁ is pyrrolidin-1-yl. In another embodiment, in the compound of formula (I), R₂ is 2-oxo-pyrrolidin-1-yl-methyl.

In another embodiment, optionally in combination with one or more features of the various embodiments described above or below throughout all the description, in the compound of formula (I), R₂ is sulfamoylphenyl, more particularly 2-sulfamoylphenyl, 3-sulfamoylphenyl, or 4-sulfamoylphenyl, even more particularly 4-sulfamoylethyl.

In another embodiment, in the compound of formula (I), R₃ is 2-methylpropyl.

In another embodiment, in the compound of formula (I) R₁ is 2-fluorophenyl or pyrrolidin-1-yl, and R₂ is 2-oxo-pyrrolidin-1-ylmethyl or 4-sulfamoylphenyl.

In another embodiment, the compound of formula (I) is selected from the group consisting of formula (II) (OCS-05), formula (III) or formula (IV).

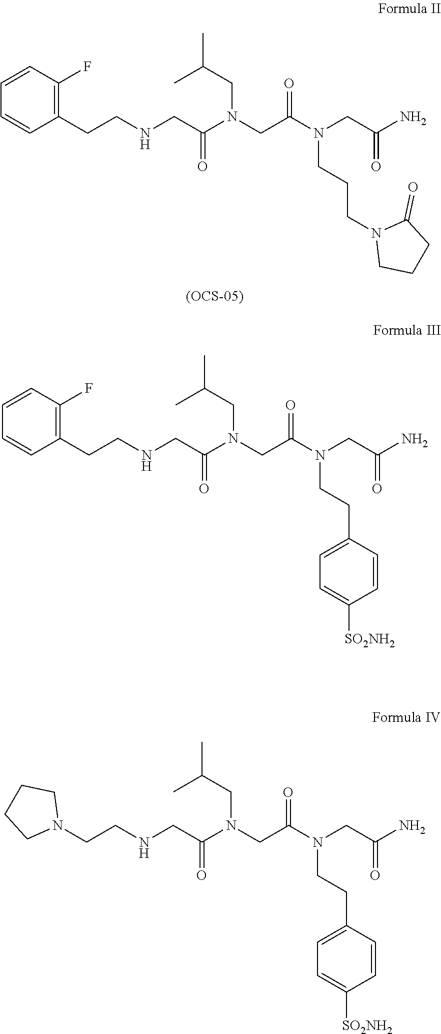 [SMILES: CC(C)CN(CC(=O)N(CCCN1CCCC1=O)CC(N)=O)C(=O)CNCCc1ccccc1F ; CC(C)CN(CC(=O)N(CCc1ccc(S(N)(=O)=O)cc1)CC(N)=O)C(=O)CNCCc1ccccc1F ; CC(C)CN(CC(=O)N(CCc1ccc(S(N)(=O)=O)cc1)CC(N)=O)C(=O)CNCCN1CCCC1]

In a preferred embodiment, a compound of formula (I) is OCS-05 (Formula II) of the following formula (II):

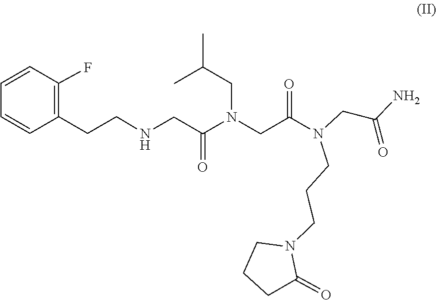 [SMILES: CC(C)CN(CC(=O)N(CCCN1CCCC1=O)CC(N)=O)C(=O)CNCCc1ccccc1F]

Compounds of formula (I), (II), (III) or (IV) can be prepared as disclosed in WO2012028959 or WO2024133860.

There is no limitation on the type of salt of the compounds of formula (I) that can be used, provided that the selected salt is pharmaceutically acceptable when used for therapeutic purposes.

The term “pharmaceutically acceptable salts”, embraces salts commonly used to form alkali metal salts and to form addition salts of free acids or free bases. The preparation of pharmaceutically acceptable salts of the compounds of formula (I) can be carried out by methods known in the art. For instance, they can be prepared from the parent compound, which contains a basic or acidic moiety, by conventional chemical methods. Generally, such salts are, for example, prepared by reacting the free acid or base forms of the compounds of formula (I) with a stoichiometric amount of the appropriate pharmaceutically acceptable base or acid in water or in an organic solvent or in a mixture of them.

The SGK2 agonist compounds, for example the compounds of formula (I), (II), (III) or (IV), and their respective salts may differ in some physical properties, but they are equivalent for the purposes of the present disclosure. The SGK2 agonist compounds may be in crystalline form either as free solvation compounds or as solvates (e.g. hydrates) and it is intended that both forms are within the scope of the present disclosure. Methods of solvation are generally known within the art. In general, the solvated forms with pharmaceutically acceptable solvents such as water, ethanol and the like are equivalent to the unsolvated form for the purposes of the disclosure.

### Methods of Treatment

The compounds of Formula (I), e.g. OCS-05 (Formula II) (Formula II), are useful to improve visual function outcome, in particular at least at 3 months after onset of optic neuropathies, for example onset of acute optic neuropathies with visual loss symptoms.

In one embodiment, the disclosure relates to a method of treating optic neuropathies with a demyelinating origin in a human subject in need thereof, said method comprising administering to said subject a therapeutically effective amount of a compound of formula (I) or (II). Demyelination may be due to inflammation, in particular inflammation of the optic nerve, and/or a neurodegenerative disorder such as multiple sclerosis.

The methods disclosed herein are particularly useful to treat patients with visual impairments after onset of optic neuropathies. Hence, for the purposes of the present disclosure, the term “treatment of visual impairments” more particularly refers to preserving, improving, or restoring long-term vision, in particular at 3 months, after the onset of acute optic neuritis with visual loss symptoms.

For example, “treatment of visual impairments” may refer to improving vision, as determined by 2.5% ETDRS Low Contrast Visual Acuity in the affected eye, for example, at 3 months or at 6 months following the start of the treatment or the onset of acute optic neuritis.

Improvement may be assessed by comparing a first population of patients treated uman with a compound of Formula (I) as disclosed herein, e.g. OCS-05 (Formula II) (Formula II), in particular in combination with an anti-inflammatory steroid treatment (such as the current standard of care for acute optic neuritis) with a second population of patients treated with only the anti-inflammatory steroid treatment, for example, as illustrated in the experimental section with the disclosed results of the clinical study.

In one embodiment, the subject treated by the methods disclosed herein is a human subject who has been diagnosed as having acute optic neuritis with visual loss symptoms. In such embodiments, the methods as disclosed herein, improve vision at about 3, about 4, about 5 or about 6 months after the start of the treatment or the onset of acute optic neuritis.

In a specific embodiment, the methods as disclosed herein, provide a statistically significant improvement in low contrast visual acuity (LCVA) up to at least month 3, 4 or 5 from administration.

In one embodiment, the methods disclosed herein provide a statistically significant difference in GCIPL (ganglion cell inner plexiform layer) thickness as a surrogate of RGC (retinal ganglion cells) preservation at least month 3, 4 or 5 from administration.

In one embodiment, the methods disclosed herein provide a statistically significant difference in RNFL (retinal nerve fiber layer) thickness as a surrogate of axon preservation up to at least month 3, 4 or 5 from administration.

In typical embodiments, a compound of Formula (I), e.g. OCS-05 (Formula II), is administered parenterally in an effective amount to a host, such as a human, in need thereof. In certain embodiments, a compound of Formula (I), e.g. OCS-05 (Formula II), is administered systemically, for example intravenously or subcutaneously. In certain embodiments, a compound of Formula (I) is administered locally, for example topically or intravitreally.

In one embodiment, the methods described herein comprises or consist of a single regimen of a once daily parenteral, for example intravenous, administration of 2-4 mg/kg (for example 3 mg/kg) of the selected compound of Formula (I), for 4-6 continuous days (for example, 5 continuous days).

In an optimal embodiment, the treatment is complete at the end of this 4-6 day regimen. In an alternative embodiment, if the day or days within the 4-6 continuous days requires that the day or days fall on a weekend or holiday, a variation can be allowed as long as the total days of the single treatment cycle does not exceed ten days.

In a preferred embodiment, the treatment should be initiated within about 10 to about 12 days of onset of symptoms. For example, in a specific embodiment, the treatment is initiated within 12 days of onset of symptoms.

The described treatment can optionally be administered on top of an anti-inflammatory steroid treatment, such as the current standard of care. This is typically one or more steroid dosages per day, at least on the days of treatment.

In a typical embodiment, the method of treatment consists of the single treatment regimen as disclosed above, which does not have to be repeated after completion, and thus is not a “discontinuous treatment regime” that requires multiple rounds of treatment with interspersed periods of no treatment. This single regimen provides convenience for the patient and results in effective treatment of the optic neuritis without undue toxicity.

In certain embodiments, the methods described herein can be used in the treatment of optic neuropathies including, but not limited to, optic neuritis (ON), acute optic neuritis (AON), anterior ischemic optic neuropathy (including non-arteritic anterior ischemic optic neuropathy), Leber disease, dominant optic atrophy, glaucoma, or toxic or traumatic tumor-related optic neuropathies.

In certain embodiments, the methods described herein can be used in the treatment of chronic relapsing inflammatory ON (CRION), autoimmune optic neuropathy, relapsing isolated ON (RION), MS-ON, neuromyelitis optica ON (NMO-ON), or single isolated ON (SION).

In certain embodiments, the human patient has demyelinating unilateral or bilateral acute optic neuritis. In certain embodiments, the human patient has anterior ischemic optic neuropathy (including non-arteritic anterior ischemic optic neuropathy), Leber disease, dominant optic atrophy, glaucoma, and toxic or traumatic tumor-related optic neuropathy. In some embodiments, the human patient has an underlying inflammation-related disease such as multiple sclerosis (MS), Neuromyelitis Optica (NMO), MOG Associated Disease (MOGAD), lupus or an inflammation-causing infection.

In certain embodiments, the methods described herein can be used in the treatment of Non-arteritic Anterior Ischemic Optic Neuropathy (NAION).

In an alternative embodiment, the method consists of a single dosage regimen which can be provided as a subcutaneous injection of 1-3 mg/kg (for example 2 mg/kg) once a day for 4-6 days (for example 5 days). Other routes of administration include intravitreal injection, which provides 1-10 μg/eye, once a day, for 4-6 days (for example 5 days) of the selected compound of Formula (I), for example, Formula (II), (III) or (IV), or an ocular topical formulation, which provides 100-500 μg/mL one drop a day of the selected compound of Formula (I), for example, Formula (II), (III) or (IV) for 4-6 days (for example 5 days).

Alternative forms of administration include intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, and tear duct injections, or through a mucus, mucin, or a mucosal barrier, in an immediate or controlled release fashion.

In certain embodiments, the dosage regimen can be provided as an intranasal dose of 15-20 mg/day. Alternatively, the dosage can be provided as a mg/kg, for example, 1-3 mg/kg (for example 2 mg/kg) once a day for 4-6 days (for example 5 days).

### Pharmaceutical Compositions

A compound of Formula (I), e.g. OCS-05 (Formula II), or a pharmaceutically acceptable salt thereof can be administered as a neat chemical but is more typically administered as a pharmaceutical composition that includes an effective amount to a host, such as a human, in need thereof. Therefore, the disclosure provides pharmaceutical compositions comprising an effective amount of a compound of Formula (I) or a pharmaceutically acceptable salt thereof together with at least one pharmaceutically acceptable carrier. The pharmaceutical composition may contain a compound of Formula (I) or a pharmaceutically acceptable salt thereof as the only active agent, or, in an alternative embodiment, the compound and at least one additional active agent.

Examples of methods to deliver a compound of Formula (I) or a pharmaceutically acceptable salt thereof include, but are not limited to, intravenous, subcutaneous, intravitreal injection, or an ocular topical formulation, or by other systemic means, in formulations containing one or more conventional pharmaceutically acceptable carriers, as appropriate. Alternative forms of administration include intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, and tear duct injections, or through a mucus, mucin, or a mucosal barrier, in an immediate or controlled release fashion. In certain embodiments, a compound of Formula I, e.g. OCS-05 (Formula II), or pharmaceutically acceptable salts thereof, is provided in a liquid dosage form, for example a liquid dosage form suitable for intravenous administration.

In certain embodiments, a systemically, for example intravenously or subcutaneously, effective amount of a compound of Formula (I) e.g. OCS-05 (Formula II), ranges from about 1 mg/kg to about 6 mg/kg of patient bodyweight. In certain embodiments, an effective amount of a compound of Formula (I) is, for example, about 1 mg/kg, about 1.5 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg or about 5 mg/kg of the compound of Formula (I), or a pharmaceutically acceptable salt thereof.

In certain embodiments, a systemically effective amount of a compound of Formula (I), e.g. OCS-05 (Formula II), ranges from about 50 mg to about 800 mg or more. In certain embodiments, an effective amount of a compound of Formula (I) is from about 50 to about 600, from about 50 mg to about 500 mg, from about 100 mg to about 400 mg, from about 150 mg to about 300 mg, from about 150 to about 250 mg. In certain embodiments, an effective amount of a compound of Formula (I) is, for example, about 140, 160, 180, 200, 220, 240, 260, 280, 300, 320, 340, 360, 380, 400, 420, 440, 460, 480, 500, 520, 540, 560, 580, or 600 mg of the compound of Formula (I), or a pharmaceutically acceptable salt thereof.

If measured as a mg/kg, then a 1 mg/kg to about 6 mg/kg of patient bodyweight may be appropriate, but is dependent on the advice of the healthcare practitioner.

In certain embodiments, a topically effective amount of a compound of Formula (I) ranges from about 200-400 μg/mL one drop a day.

In certain embodiments, an intravitreally effective amount of a compound of Formula (I) ranges from about 1-5 μg/eye.

In certain embodiments, a intravitreally effective amount of a compound of Formula (I), e.g OCS-05 (Formula II), ranges from about 1 μg to about 10 μg/eye or more. In certain embodiments, the intravitreal dose is between about 2 μg/eye and 15 μg/eye or more. In certain embodiments, the intravitreal dose is about 1 μg/eye to about 14 μg/eye, from about 1 μg/eye to about 12 μg/eye, from about 1 μg/eye to about 10 μg/eye, from about 1 μg/eye to about 8 μg/eye, from about 1 μg/eye to about 7 μg/eye, from about 1 μg/eye to about 6 μg/eye, from about 1 μg/eye to about 5 μg/eye, or from about 2 μg/eye to about 5 μg/eye. In certain embodiments the intravitreal injection dose is about 1, 2, 3, 4, 5, 6, 7, 8, 9, 10, 11, 12, 13, 14, or 15 μg/eye of the compound of Formula (I), or a pharmaceutically acceptable salt thereof.

### Intravenous Administration

In certain embodiments, an effective amount of the compound of Formula (I), e.g. OCS-05 (Formula II), or a pharmaceutically acceptable salt thereof is administered intravenously to a host such as a human in need thereof. Typically, if administered intravenously, the compound will be formulated in a liquid dosage form for intravenous injection, such as a buffered solution. Non-limiting examples of solutions for intravenous injection include phosphate buffered solution and saline buffered solution. In certain embodiments the solution is buffered with multiple salts.

In certain embodiments, an effective amount of a compound of Formula (I), e.g. OCS-05 (Formula II), is, for example, about 1 mg/kg, about 1.5 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg or about 5 mg/kg of the compound of Formula (I), or a pharmaceutically acceptable salt thereof.

In certain embodiments, the pharmaceutical composition is a lyophilized powder to be reconstituted for intravenous infusion. In other embodiments, the pharmaceutical composition is provided in a solution suitable for intravenous injection.

For example, the pharmaceutical composition is a lyophilized powder of a compound of Formula (I) or a pharmaceutically acceptable salt thereof, for example Formula (II), to be reconstituted with an appropriate volume of sterile saline solution for intravenous administration. In certain embodiments, the pharmaceutical composition is a lyophilized powder of a compound of Formula (I), e.g. OCS-05 (Formula II), or a pharmaceutically acceptable salt thereof, in combination with one or more excipients to be reconstituted with an appropriate volume of sterile saline solution for intravenous administration.

In one embodiment, the pharmaceutical composition comprises a compound of Formula (I) or a pharmaceutically acceptable salt thereof, for example Formula (II), and a buffer agent, for example citric acid monohydrate and trisodium citrate dihydrate.

In certain embodiments, the pharmaceutical composition is a sterile saline solution comprising a compound of Formula (I) or a pharmaceutically acceptable salt thereof, for example Formula (II), for intravenous administration.

### Topical Administration

In certain embodiments, pharmaceutical compositions comprising a compound of Formula (I), for example, OCS-05 (Formula II) or a pharmaceutically acceptable salt thereof can also be administered to the patient by a topical route, e.g., eye drop, eye gel, eye ointment, or the like. In certain embodiments a locally effective amount of a compound of Formula (I) or a pharmaceutically acceptable salt thereof is administered to the eye of the host, such as a human, in need thereof. In certain embodiments, the locally effective amount of a compound of Formula (I) is from about 100-500 μg/ml, one drop a day, for example 200 μg/ml one drop a day.

### Intravitreal Administration

In certain embodiments, pharmaceutical compositions comprising a compound of Formula (I), for example, OCS-05 (Formula II) or a pharmaceutically acceptable salt thereof can also be administered in an effective amount to a host such as a human in need thereof intravitreally. In certain embodiments, a locally effective amount of compound of Formula (I), or a pharmaceutically acceptable salt thereof is administered intravitreally. In certain embodiments, the locally effective amount of a compound of Formula (I) is from about 1-10 μg/eye, for example 5 μg/eye.

In certain embodiments, intravitreal injection of a pharmaceutical formulation comprising an effective amount of a compound of Formula (I) or a pharmaceutically acceptable salt thereof includes the step of piercing the eye of the host with a syringe and needle containing the formulation and injecting the formulation (for example typically less than or equal to about 100 microliters; about 40, 50, 55, 56, 57, 57.1, 58, 60, 70 or 75 microliters) into the vitreous of the eye (e.g., with a sufficient volume as to deliver a locally effective amount of compound of Formula (I) or a pharmaceutically acceptable salt thereof. Optionally, the method includes the steps of administering a local anesthetic (e.g., proparacaine, lidocaine or tetracaine), an antibiotic (e.g., a fluoroquinolone), antiseptic (e.g., povidone-iodine) and/or a pupil dilating agent to the eye being injected. In an embodiment, a sterile field around the eye to be injected is established before the injection. In an embodiment, following intravitreal injection, the subject is monitored for elevations in intraocular pressure and/or blood pressure. In an embodiment, the other eye is also injected by the same procedure.

### Subcutaneous Administration

In certain embodiments, an effective amount of a compound of Formula (I), e.g. OCS-05 (Formula II), or a pharmaceutically acceptable salt thereof is administered subcutaneously.

A subcutaneous administration can be in a standard mg dosage form or alternatively measured on a mg/kg basis based on the weight of the host.

In certain embodiments, a systemically, for example intravenously or subcutaneously, effective amount of a compound of Formula (I), e.g. OCS-05 (Formula II), ranges from about 1 mg/kg to about 4 mg/kg of patient bodyweight. In certain embodiments, an effective amount of a compound of Formula (I) is, for example, about 1 mg/kg, about 1.5 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, or about 4 mg/kg of the compound of Formula (I), or a pharmaceutically acceptable salt thereof.

In a typical embodiment, the volume of a pharmaceutical composition for subcutaneous administration is less than about 3.0, 2.5, 2.0, 1.75, 1.5, 1.4, 1.3, 1.2, 1.1, or 1.0 mL (corresponds to 2-6 mg/kg in a 1.0 mL-1.5 mL subQ dose).

In certain embodiments, the liquid dosage form of a compound described herein for subcutaneous administration is formulated in about 1.5, 1.4, 1.3, 1.2, 1.1, or 1.0 mL. In certain embodiments, the liquid dosage form of a compound described herein for subcutaneous administration comprises the compound at a concentration of about 50 mg/mL to about 500 mg/mL, from about 75 mg/mL to about 400 mg/mL, from about 100 mg/mL to about 300 mg/mL, or from about 150 mg/mL to about 300 mg/mL. In certain embodiments, the liquid dosage form of a compound described herein for subcutaneous administration comprises the compound at a concentration of about 100, 120, 140, 160, 180, 200, 220, 240, 260, 280, or 300 mg/mL.

Typically, the compound will be formulated in a liquid dosage form for subcutaneous injection comprising the selected compound and one or more excipients. Examples of excipients suitable for use in a subcutaneous formulation include but are not limited to a buffer, tonicity agent, preservative, and/or stabilizer. Non-limiting examples of solutions for subcutaneous injection include phosphate buffered solution and saline buffered solution. In certain embodiments the solution is buffered with multiple salts. In certain embodiments, the solution for subcutaneous administration comprises an amino acid or a salt thereof. In certain embodiments, the amino acid is arginine, histidine, phenylalanine, or ornithine. In certain embodiments, the solution for subcutaneous injection comprises an anionic excipient, including but not limited to benzenesulfonic acid, pyridoxine, and thiamine.

In certain embodiments, the pharmaceutical composition provides a compound of Formula (I), e.g. OCS-05 (Formula II), or a pharmaceutically acceptable salt thereof in an immediate release fashion. In certain embodiments, the pharmaceutical composition provides a compound of Formula (I) or (II) or a pharmaceutically acceptable salt thereof in a controlled release fashion. In certain embodiments, the pharmaceutical composition provides a compound of Formula (I) as a long-acting injectable (LAI). LAIs offer multiple advantages for patients. For instance, these formulations can provide a more consistent drug concentration in the body, which can lead to fewer side effects, improved symptom control and faster healing.

A medical device may be used for the self-administration of a compound of Formula (I), e.g. OCS-05 (Formula II), or a pharmaceutically acceptable salt thereof to the host in need thereof such as a human, for example a prefilled syringe, an injection pen, or an autoinjector. In certain embodiments, a predetermined dose of a compound of formula (I) is self-administered with a medical device, for example prefilled syringe, an injection pen, or an autoinjector including but not limited to a multidose autoinjector, a disposable autoinjector, a reusable autoinjector, and a variable dose autoinjector. In certain embodiments, the medical device is prefilled with an effective amount of a compound of Formula (I), or a pharmaceutically acceptable salt thereof, optionally in a pharmaceutically acceptable carrier. In certain embodiments, the medical device contains about 1.5, 1.4, 1.3, 1.2, 1.1, or 1.0 mL of a subcutaneous formulation as described herein. In certain embodiments, the medical device contains from about 50 mg to about 800 mg or more of a compound of Formula (I), e.g. OCS-05 (Formula II), or a pharmaceutically acceptable salt thereof. In certain embodiments, the medical device contains about 50 to about 600, from about 50 mg to about 500 mg, from about 100 mg to about 400 mg, from about 150 mg to about 300 mg, from about 150 to about 250 mg of a compound of Formula (I), e.g. OCS-05 (Formula II), or a pharmaceutically acceptable salt thereof.

In certain embodiments, the medical device contains, for example, about 140, 160, 180, 200, 220, 240, 260, 280, 300, 320, 340, 360, 380, 400, 420, 440, 460, 480, 500, 520, 540, 560, 580, or 600 mg of a compound of Formula (I), or a pharmaceutically acceptable salt thereof.

### Intranasal Administration

In other embodiments, a compound of Formula (I) or a pharmaceutically acceptable salt thereof, is provided in an effective amount of a pharmaceutical composition suitable for intranasal administration to a host such as a human in need thereof.

In certain embodiments, the intranasal formulation comprises one or more excipients such as cyclodextrins, permeation enhancers, surfactants, bile salts, lipids, cyclodextrins, polymers, or tight junction modifiers. In certain embodiments, the intranasal formulation comprises an excipient selected from poloxamers (407, 188), CS, castor oil, polyoxyl 40 hydrogenated castor oil, 1,2-propanediol, Ringer's solution, Na-CMC, glyceryl behenate glyceryl palmitostearate, glyceryl monostearate, precirol, carrageenan, soybean phospholipids, cholesterol, tween 80, sodium caprate, sodium cholate, sodium caprate, β-cyclodextrin, HPMC E5, PEG 6000, PEG 4000, polyethylene, HPMCK4M, HPMC, borneol, benzalkonium chloride, NaCl, Carbopol® 974P NF, PEG 400, benzalkonium chloride, dexpanthenol, and triethanolamine.

### Additional Routes of Administration

In certain embodiments, an effective amount of a compound of Formula (I) or a pharmaceutically acceptable salt thereof, is provided in a pharmaceutical composition suitable for administration to the host such as a human by ocular, intraocular, intrachoroidal, or subconjunctival injection.

In other embodiments, the compound of Formula (I) or a pharmaceutically acceptable salt thereof, is provided in a pharmaceutical composition suitable for intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, or tear duct injection administration.

In certain embodiments, the compound of Formula (I) or a pharmaceutically acceptable salt thereof, is provided in a pharmaceutical composition suitable for administration through a mucus, mucin, or a mucosal barrier.

### Pharmaceutical Excipients and Components

Compositions as disclosed herein can be useful in formulation of parenteral controlled release or sustained release systems (both systems leading to a many-fold reduction in number of administrations). Also included, are controlled release and sustained release systems administered subcutaneously. Without limiting the scope of the disclosure, examples of useful controlled release system and compositions are hydrogels, oleaginous gels, liquid crystals, polymeric micelles, microspheres, nanoparticles. Methods to produce controlled release systems useful for compositions of the current disclosure include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Carriers include excipients and/or diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution (HA, 5 mg/mL in PBS), citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol. Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

In certain embodiments, the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. In certain embodiments, the isotoncity agent is propyleneglycol. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In certain embodiments, the sugar additive is sucrose. Sugar alcohol is defined as a C4-C8 hydrocarbon having at least one-OH group and includes, for example, mannitol, sorbitol, inositol, galactitol, dulcitol, xylitol, and arabitol. In certain embodiments, the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods disclosed herein. In certain embodiments, the sugar or sugar alcohol concentration is between about 1 mg/ml and about 150 mg/ml. In certain embodiments, the isotonic agent is present in a concentration from 1 mg/ml to 50 mg/ml. In certain embodiments, the isotonic agent is present in a concentration from 1 mg/ml to 7 mg/ml. In certain embodiments, the isotonic agent is present in a concentration from 5 mg/ml to 7 mg/ml. In certain embodiments, the isotonic agent is present in a concentration from 8 mg/ml to 24 mg/ml. In certain embodiments, the isotonic agent is present in a concentration from 25 mg/ml to 50 mg/ml. Each one of these specific isotonic agents constitutes an alternative embodiment. For convenience reference is made to Remington: The Science and Practice of Pharmacy, 19th edition, 1995.

In addition to the active compounds or their salts, the pharmaceutical formulations can contain other additives, such as pH-adjusting additives (i.e. a buffer). In particular, useful pH-adjusting agents include acids, such as hydrochloric acid, bases or buffers, such as sodium lactate, sodium acetate, sodium phosphate, sodium citrate, sodium borate, or sodium gluconate. In a certain embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof.

### Salts

The term “pharmaceutically acceptable salt” as used herein refers to a salt of the described compound which is, within the scope of sound medical judgment, suitable for administration to a host such as a human without undue toxicity, irritation, allergic response, and the like, commensurate with a reasonable benefit/risk ratio, and effective for its intended use. Thus, the term “pharmaceutically acceptable salt” refers to the relatively non-toxic, inorganic and organic acid addition salts of the presently disclosed compounds. These salts can be prepared during the final isolation and purification of the compounds or by separately reacting the purified compound in its free form with a suitable organic or inorganic acid and then isolating the salt thus formed. Basic compounds are capable of forming a wide variety of different salts with various inorganic and organic acids. Acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents. Pharmaceutically acceptable base addition salts may be formed with a metal or amine, such as alkali and alkaline earth metal hydroxide, or an organic amine. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

### Combination Therapy

The compound of Formula (I), e.g. OCS-05 (Formula II), or pharmaceutically acceptable salt as described herein can be used as a monotherapy, but is can be administered as a combination therapy, for example on top of a standard of care. In certain embodiments, a method of treating optic neuropathy in a patient in need thereof comprises administering an effective amount of a compound of Formula (I) or Formula (II), for example, with a single regimen of a once daily parenteral such as intravenous administration of 2-4 mg/kg (for example 3 mg/kg) of the selected compound of Formula (I), for 4-6 continuous days (for example, 5 continuous days) in combination with one or more additional therapeutic agents.

As used herein, the term “combination therapy” is intended to include treatment regimens in which the agents are not necessarily administered by the same route of administration or at the same time.

In certain embodiments, the method of treatment described herein is administered in combination with standard of care, which is typically a corticosteroid (steroid). In certain embodiments, the method of treatment described herein is administered in combination with an intravenously administered steroid. In other embodiments, the method of treatment described herein is administered in combination with an orally administered steroid. In certain embodiments, the steroid is selected from dexamethasone, hydrocortisone, methylprednisolone, and prednisone. In certain embodiments, the steroid is prednisone or methylprednisolone. In certain embodiments, the steroid is prednisone. In certain embodiments, the steroid is methylprednisolone.

A steroid treatment regimen typically includes parenteral such as intravenous steroid treatment, for example methylprednisolone, optionally followed by oral steroid treatment, for example prednisone. In certain embodiments, the standard of care intravenous steroid treatment comprises administering from about 500 mg/day to about 1,500 mg/day of a steroid. In certain embodiments, the standard of care intravenous steroid treatment comprises administering from about 500, 600, 700, 800, 900, 1000, 1100, 1200, 1300, 1400, or 1500 mg/day of a steroid. In certain embodiments, the intravenous steroid treatment comprises about 1,000 mg/day of a steroid. In certain embodiments, the intravenous steroid treatment is optionally followed by an oral steroid treatment. In certain embodiments the oral steroid treatment comprises about or at least about 1, 2, 3, 4, 5, 6, 7, 8, 9, 10, 11, 12, 13, 14, 21, or 28 days of oral steroid treatment. In certain embodiments the oral steroid treatment comprises about or at least about 7, 8, 9, 10, 11, 12, 13, or 14 days of oral steroid treatment. In certain embodiments the oral steroid treatment comprises about or at least about 11 days of oral steroid treatment.

In certain embodiments, a method of treating optic neuropathy in a patient in need thereof comprises a single regimen of a once daily intravenous administration of 2-4 mg/kg (for example 3 mg/kg) of the selected compound of Formula (I), e.g. OCS-05 (Formula II), for 4-6 continuous days (for example, 5 continuous days) in combination with a steroid for three to five days.

In certain embodiments, the compound of Formula (I), e.g. OCS-05 (Formula II), or a pharmaceutically acceptable salt thereof, and the steroid are administered starting on the same day. In certain embodiments, administration of the steroid starts 1, 2, 3, 4, or 5 days after the first administration of the compound of Formula (I), or pharmaceutically acceptable salt thereof.

In certain embodiments, a method of treating optic neuropathy in a patient in need thereof comprises a single regimen of a once daily intravenous administration of 2-4 mg/kg (for example 3 mg/kg) of the selected compound of Formula (I), e.g. OCS-05 (Formula II), for 4-6 continuous days (for example, 5 continuous days) in combination with three days of steroid treatment.

In certain embodiments, a method of treating optic neuropathy in a patient in need thereof comprises a single regimen of a once daily intravenous administration of 2-4 mg/kg (for example 3 mg/kg) of the selected compound of Formula (I), e.g. OCS-05 (Formula II), for 4-6 continuous days (for example, 5 continuous days) in combination with four days of steroid treatment.

In certain embodiments, a method of treating optic neuropathy in a patient in need thereof comprises a single regimen of a once daily intravenous administration of 2-4 mg/kg (for example 3 mg/kg) of the selected compound of Formula (I), e.g. OCS-05 (Formula II), for 4-6 continuous days (for example, 5 continuous days) in combination with five days of steroid treatment.

In certain embodiments, a method of treating optic neuropathy in a patient in need thereof comprises a single regimen of a once daily intravenous administration of 2-4 mg/kg (for example 3 mg/kg) of the selected compound of Formula (I), e.g. OCS-05 (Formula II), for 4-6 continuous days (for example, 5 continuous days) in combination with three, four, or five days of intravenous steroid treatment, optionally followed by about or at least about 1, 2, 3, 4, 5, 6, 7, 8, 9, 10, 11, 12, 13, 14, 21, or 28 days of oral steroid treatment.

In certain embodiments, a method of treating optic neuropathy in a patient in need thereof comprises a single regimen of a once daily intravenous administration of 2-4 mg/kg (for example 3 mg/kg) of the selected compound of Formula (I), e.g. OCS-05 (Formula II), for 4-6 continuous days (for example, 5 continuous days) in combination with about 800 to about 1,200 mg/day of an intravenously administered steroid, for example methylprednisolone.

In certain embodiments, a method of treating optic neuropathy in a patient in need thereof comprises a single regimen of a once daily intravenous administration of 2-4 mg/kg (for example 3 mg/kg) of the selected compound of Formula (I), e.g. OCS-05 (Formula II), for 4-6 continuous days (for example, 5 continuous days) in combination with about 800 to about 1,200 mg/day of an intravenously administered steroid, for example methylprednisolone followed by 1 mg/kg/d oral prednisone for about 1, 2, 3, 4, 5, 6, 7, 8, 9, 10, 11, 12, 13, 14, 21, or 28 days.

In certain embodiments, a method of treating optic neuropathy in a patient in need thereof comprises a single regimen of a once daily intravenous administration of 2-4 mg/kg (for example 3 mg/kg) of the selected compound of Formula (I), for 4-6 continuous days (for example, 5 continuous days) in combination with 1,000 mg/day of intravenously administered steroid, for example methylprednisolone followed by 1 mg/kg/d oral prednisone for about 7, 8, 9, 10, or 11 days.

In certain embodiments, a method of treating optic neuropathy in a patient in need thereof comprises a single regimen of a once daily intravenous administration of 2-4 mg/kg (for example 3 mg/kg) of the selected compound of Formula (I), e.g. OCS-05 (Formula II), for 4-6 continuous days (for example, 5 continuous days) in combination with an immunomodulator, for example intravenous immunoglobulin (IVIG). In certain embodiments, the IVIG is administered at about 0.1, 0.2, 0.3, 0.4, or 0.5 g/kg, for example 0.4 g/kg.

In certain embodiments, the method of treatment described herein is administered in combination with an immunomodulatory drug, for example as used in standard of care for multiple sclerosis, NMO or MOGAD, which may be for example disease modifying therapies, including fingolimod, dimethyl fumarate, teriflunomide, interferon beta, glatiramer acetate, natalizumab, alemtuzumab or ocrelizumab. In certain embodiments, the method of treatment described herein is administered in combination with an intravenously administered steroid and immunomodulatory drugs used as disease modifying therapies for multiple sclerosis, NMO or MOGAD.

### SPECIFIC EMBODIMENTS

The present disclosure provides the specific methods of use of the compound of Formula I:
1. A method for the treatment of optic neuropathy, comprising administering to a human in need thereof an effective amount of a compound of Formula I or a pharmaceutically acceptable salt thereof, with a single treatment regimen of a once daily administration of 2-5 mg/kg for 4, 5, or 6 continuous days, wherein Formula I is

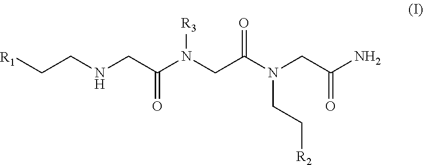

and
R₁ is phenyl substituted with halogen or trifluoromethyl, and further optionally substituted with one or two substituents selected from the group consisting of halogen, (C₁₋₆) alkyl, (C₁₋₆) alkoxy, and halo(C₁₋₆)alkyl; or
R₁ is pyrrolidin-1-yl;
R₂ is

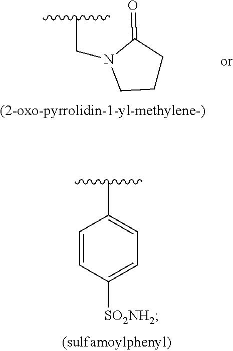 [SMILES: *CN1CCCC1=O ; *c1ccc(S(N)(=O)=O)cc1]

and
R₃ is selected from ethyl, propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl.
2. A method for the treatment of optic neuropathy, comprising administering to a human in need thereof an effective amount of a compound of Formula I or a pharmaceutically acceptable salt thereof, with a single regimen 4, 5, or 6 daily administrations spread over not longer than ten days wherein Formula I is

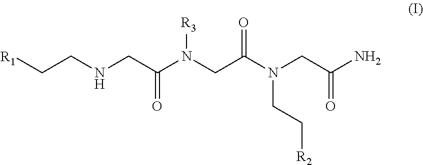

and
R₁ is phenyl substituted with halogen or trifluoromethyl, and further optionally substituted with one or two substituents selected from the group consisting of halogen, (C₁₋₆) alkyl, (C₁₋₆) alkoxy, and halo(C₁₋₆)alkyl; or
R₁ is pyrrolidin-1-yl;
R₂ is

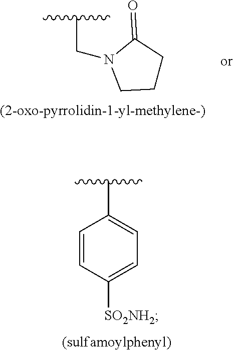 [SMILES: *CN1CCCC1=O ; *c1ccc(S(N)(=O)=O)cc1]

and
R₃ is selected from ethyl, propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl.
3. The method of embodiment 1 or 2, wherein the compound of Formula 1 is selected from

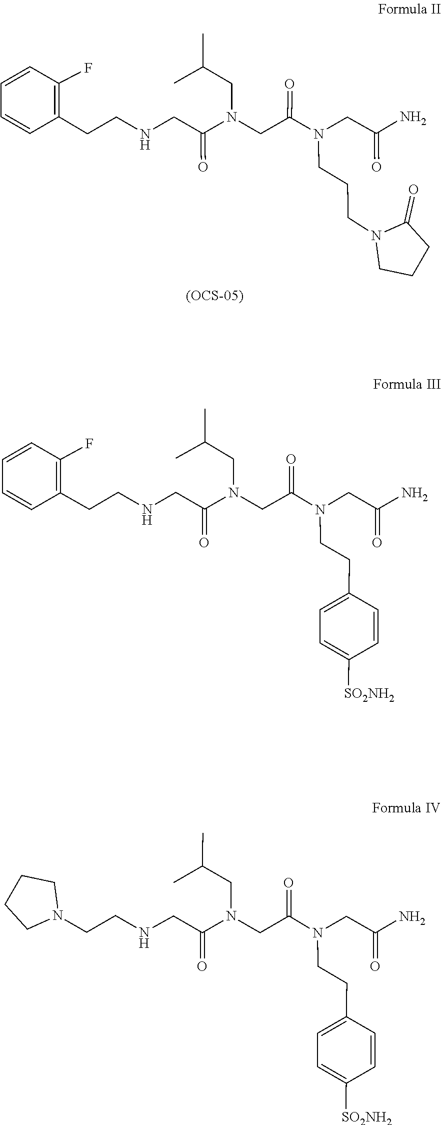 [SMILES: CC(C)CN(CC(=O)N(CCCN1CCCC1=O)CC(N)=O)C(=O)CNCCc1ccccc1F ; CC(C)CN(CC(=O)N(CCc1ccc(S(N)(=O)=O)cc1)CC(N)=O)C(=O)CNCCc1ccccc1F ; CC(C)CN(CC(=O)N(CCc1ccc(S(N)(=O)=O)cc1)CC(N)=O)C(=O)CNCCN1CCCC1]

or a pharmaceutically acceptable salt thereof.
4. The method of embodiment 3, wherein the compound of Formula I is Formula II, or a pharmaceutically acceptable salt thereof.
5 The method of embodiment 3, wherein the compound of Formula I is Formula III or a pharmaceutically acceptable salt thereof.
6. The method of embodiment 3, wherein the compound of Formula I is Formula IV or a pharmaceutically acceptable salt thereof.
7. The method of any one of embodiments 1-6, wherein the compound of Formula I is administered via an intravenous injection.
8. The method of any one of embodiments 1-6, wherein the compound of Formula I is administered via an intravitreal injection.
9. The method of any one of embodiments 1-6, wherein the compound is administered via subcutaneous injection.
10. The method of any one of embodiments 1-6, wherein the compound is administered via a topical formulation.
11. The method of any one of embodiments 1-6, wherein the compound is administered via intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, or tear duct injection, or through a mucus, mucin, or a mucosal barrier, in an immediate or controlled release fashion.
12. The method of any one of embodiments 1-11, wherein the treatment is initiated within 12 days of onset of symptoms.
13. The method of any one of embodiments 1-12, wherein the treatment is administered on top of standard of care.
14. The method of embodiment 13, wherein the standard of care is an anti-inflammatory steroid.
15. The method of any one of embodiments 1-14, wherein the optic neuropathy is acute optic neuritis.
16. The method of any one of embodiments 1-14, wherein the optic neuropathy is anterior ischemic optic neuropathy, particularly a Non-arteritic Anterior Ischemic Optic Neuropathy,
17. The method of any one of embodiments 1-14, wherein the optic neuropathy is caused by multiple sclerosis.
18. The method of any one of embodiments 1-14, wherein the optic neuropathy is caused by a disorder selected from Leber disease, dominant optic atrophy, glaucoma, and toxic or traumatic tumor-related optic neuropathy.
19. The method of any one of embodiments 1-14, wherein the optic neuropathy is caused by Neuromyelitis Optica (NMO), MOG Associated Disease (MOGAD), lupus or an inflammation-causing infection.
20. The method of any one of embodiments 1-19, wherein 3 mg/kg of the compound is administered.
21. The method of any one of embodiments 1-19, wherein the compound is administered for five days.
22. The compound of any one of embodiments 1-19, wherein 3 mg/kg of the compound is administered for five days.
23. The method of any one of embodiments 1-2 and 7-19, wherein the R₁ is fluorophenyl
24. The method of any one of embodiments 1-2 and 7-19, wherein R₁ is 2-fluorophenyl.
25. The method of any one of embodiments 1-2 and 7-19, wherein R₁ is fluorophenyl which is further substituted with one or two substituents selected from the group consisting of halogen, (C₁₋₆)alkyl, (C₁₋₆)alkoxy, and halo(C₁₋₆)alkyl.
26. The method of any one of embodiments 1-2 and 7-19, wherein R₁ is fluorophenyl which is further substituted with one or two substituents selected from the group consisting of halogen, (C₁₋₄)alkyl, (C₁₋₄)alkoxy, and halo(C₁₋₄)alkyl.
27. The method of any one of embodiments 1-2 and 7-19, wherein R₁ is fluorophenyl which is further substituted with one or two substituents selected from the group consisting of halogen, methyl, ethyl, propyl, isopropyl, methoxy, ethoxy, fluoromethyl, and trifluoromethyl.
28. The method of any one of embodiments 1-2 and 7-19, wherein R₁ is chlorophenyl.
29. The method of any one of embodiments 1-2 and 7-19, wherein R₁ is selected from 2-chlorophenyl, 3-chlorophenyl and 4-chlorophenyl.
30. The method of any one of embodiments 1-2 and 7-19, wherein R₁ is 2-chlorophenyl.
31. The method of any one of embodiments 1-2 and 7-19, wherein R₁ is chlorophenyl which is further substituted with one or two substituents selected from the group consisting of halogen, (C₁₋₆)alkyl, (C₁₋₆)alkoxy, and halo(C₁₋₆)alkyl
32. The method of any one of embodiments 1-2 and 7-19, wherein R₁ is fluorophenyl which is further substituted with one or two substituents selected from the group consisting of halogen, (C₁₋₄)alkyl, (C₁₋₄)alkoxy, and halo(C₁₋₄)alkyl.
33. The method of any one of embodiments 1-2 and 7-19, wherein R₁ is bromophenyl.
34. The method of any one of embodiments 1-2 and 7-19, wherein R₁ is selected from 2-bromophenyl, 3-bromophenyl and 4-bromophenyl,
35. The method of any one of embodiments 1-2 and 7-19, wherein R₁ is 2-bromophenyl.
36. The method of any one of embodiments 1-2 and 7-19, wherein R₁ is bromophenyl which is further substituted with one or two substituents selected from the group consisting of halogen, (C₁₋₆)alkyl, (C₁₋₆)alkoxy, and halo(C₁₋₆)alkyl
37. The method of any one of embodiments 1-2 and 7-19, wherein R₁ is bromophenyl which is further substituted with one or two substituents selected from the group consisting of halogen, (C₁₋₄)alkyl, (C₁₋₄)alkoxy, and halo(C₁₋₄)alkyl.
38. The method of any one of embodiments 1-2 and 7-19, wherein R₁ is bromophenyl which is further substituted with one or two substituents selected from the group consisting of halogen, methyl, ethyl, propyl, isopropyl, methoxy, ethoxy, fluoromethyl, and trifluoromethyl.
39. The method of any one of embodiments 1-2 and 7-19, wherein R₁ is iodophenyl.
40. The method of any one of embodiments 1-2 and 7-19, wherein R₁ is 2-iodophenyl, 3-iodophenyl or 4-iodophenyl.
41. The method of any one of embodiments 1-2 and 7-19, wherein R₁ is 2-iodophenyl.
42. The method of any one of embodiments 1-2 and 7-19, wherein R₁ is iodophenyl which is substituted with one or two substituents selected from the group consisting of halogen, (C₁₋₆)alkyl, (C₁₋₆)alkoxy, and halo(C₁₋₆)alkyl.
43. The method of any one of embodiments 1-2 and 7-19, wherein R₁ is iodophenyl which is further substituted with one or two substituents selected from the group consisting of halogen, (C₁₋₄)alkyl, (C₁₋₄)alkoxy, and halo(C₁₋₄)alkyl.
44. The method of any one of embodiments 1-2 and 7-19, wherein R₁ is iodophenyl which is further substituted with one or two substituents selected from the group consisting of halogen, methyl, ethyl, propyl, isopropyl, methoxy, ethoxy, fluoromethyl, and trifluoromethyl.
45. The method of any one of embodiments 1-2 and 7-19, wherein R₁ is trifluoromethylphenyl.
46. The method of any one of embodiments 1-2 and 7-19, wherein R₁ is 2-trifluoromethylphenyl, 3-trifluoromethylphenyl or 4-trifluoromethylphenyl.
47. The method of any one of embodiments 1-2 and 7-19, wherein R₁ is 2-trifluoromethylphenyl.
48. The method of any one of embodiments 1-2 and 7-19, wherein R₁ is trifluoromethylphenyl which is further substituted with one or two substituents selected from the group consisting of halogen, (C₁₋₆)alkyl, (C₁₋₆)alkoxy, and halo(C₁₋₆)alkyl.
49. The method of any one of embodiments 1-2 and 7-19, wherein R₁ is trifluoromethylphenyl which is further substituted with one or two substituents selected from the group consisting of halogen, (C₁₋₄)alkyl, (C₁₋₄)alkoxy, and halo(C₁₋₄)alkyl.
50. The method of any one of embodiments 1-2 and 7-19, wherein R₁ is trifluoromethylphenyl which is further substituted with one or two substituents selected from the group consisting of halogen, methyl, ethyl, propyl, isopropyl, methoxy, ethoxy, fluoromethyl, and trifluoromethyl.
51. The method of any one of embodiments 1-2 and 7-19, wherein R₁ is pyrrolidin-1-yl.
52. The method of any one of embodiments 1-2 and 7-19, wherein R₂ is 2-oxo-pyrrolidin-1-yl-methyl.
53. The method of any one of embodiments 1-2 and 7-19, wherein R₂ is sulfamoylphenyl.
54. The method of any one of embodiments 1-2 and 7-19, wherein R₂ is 2-sulfamoylphenyl, 3-sulfamoylphenyl, or 4-sulfamoylphenyl.
55. The method of any one of embodiments 1-2 and 7-19, wherein R₂ is 4-sulfamoylethyl.
56. The method of any one of embodiments 1-2 and 7-19, wherein R₃ is 2-methylpropyl.
57. The method of any one of embodiments 1-2 and 7-19, wherein R₁ is 2-fluorophenyl or pyrrolidin-1-yl, and R₂ is 2-oxo-pyrrolidin-1-ylmethyl or 4-sulfamoylphenyl.
58. The method of any one of embodiments 1-54, wherein the single treatment regimen provides a statistically significant difference in GCIPL (ganglion cell inner plexiform layer) thickness as a surrogate of RGC (retinal ganglion cells) preservation at least month 3 from administration.
59. The method of any one of embodiments 1-54, wherein the single treatment regimen provides a statistically significant difference in RNFL (retinal nerve fiber layer) thickness as a surrogate of axon preservation up to at least month 3 from administration.
60. The method of any one of embodiments 1-54, wherein the single treatment regimen provides a statistically significant improvement in low contrast visual acuity (LCVA) up to at least month 3 from administration.
61. The method of any one of embodiments 55-57, wherein the single treatment regimen results in drug-related serious adverse events or adverse events leading to drug withdrawal.
62. The method of any one of embodiments 55-57, wherein the single treatment regimen results in a lower incidence of adverse events related to multiple sclerosis relapses
63. The method of any one of embodiments 55-57, wherein the single treatment regimen results in a lower incidence of adverse events related to worsening of CNS inflammatory disorders.
64. The method of any one of embodiments 55-57, wherein the single treatment regimen results in no difference in percent of patients with abnormal electrocardiogram (ECG) events between dosed patients and placebo patients.
65. The method of any one of embodiments 1 or 2, wherein the human patient is diagnosed with an optic neuropathy that is demyelinating acute optic neuritis.
66 The method of any one of embodiments 1-62, wherein the onset of visual loss symptoms occurs within the last 12 days before treatment begins.
67. The method of any one of embodiments 1-62, wherein the human patient does not have known neuromyelitis optica with autoantibodies against aquaporin-4 (AQP4-Abs).
68. A compound of Formula I:

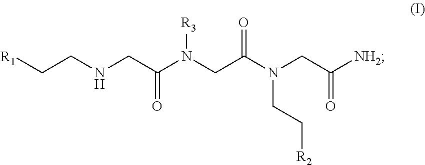

or a pharmaceutically acceptable salt thereof, wherein:
R₁ is phenyl substituted with halogen or trifluoromethyl, and further optionally substituted with one or two substituents selected from the group consisting of halogen, (C₁₋₆) alkyl, (C₁₋₆) alkoxy, and halo(C₁₋₆)alkyl; or
R₁ is pyrrolidin-1-yl;
R₂ is

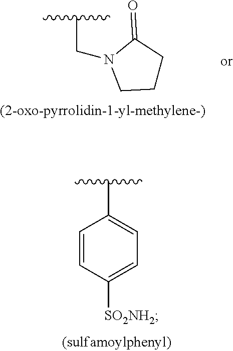 [SMILES: *CN1CCCC1=O ; *c1ccc(S(N)(=O)=O)cc1]

and
R₃ is selected from ethyl, propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl;
for use in the treatment of optic neuropathy, comprising administering to a human in need thereof an effective amount of the compound or a pharmaceutically acceptable salt thereof.
69. A compound of Formula I:

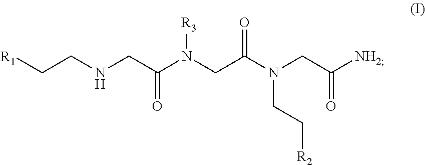

or a pharmaceutically acceptable salt thereof, wherein:
R₁ is phenyl substituted with halogen or trifluoromethyl, and further optionally substituted with one or two substituents selected from the group consisting of halogen, (C₁₋₆) alkyl, (C₁₋₆) alkoxy, and halo(C₁₋₆)alkyl; or
R₁ is pyrrolidin-1-yl;
R₂ is

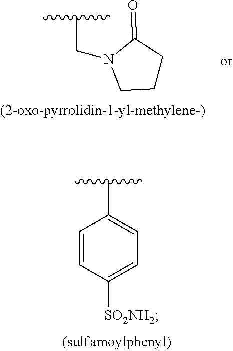 [SMILES: *CN1CCCC1=O ; *c1ccc(S(N)(=O)=O)cc1]

and
R₃ is selected from ethyl, propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl;
for use in a method for treating visual impairments in a human subject having an optic neuropathy, said method comprising administering an effective amount of said compound of Formula (I), or a pharmaceutically acceptable salt thereof, in said subject.
70. The compound for use of embodiment 68 or 69, wherein an effective amount of the compound or a pharmaceutically acceptable salt thereof, is administered in a single treatment regimen of a once daily administration of 2-5 mg/kg for 4, 5 or 6 continuous days.
71. The compound for use of embodiment 68, 69, or 70, wherein the compound of Formula 1 is selected from

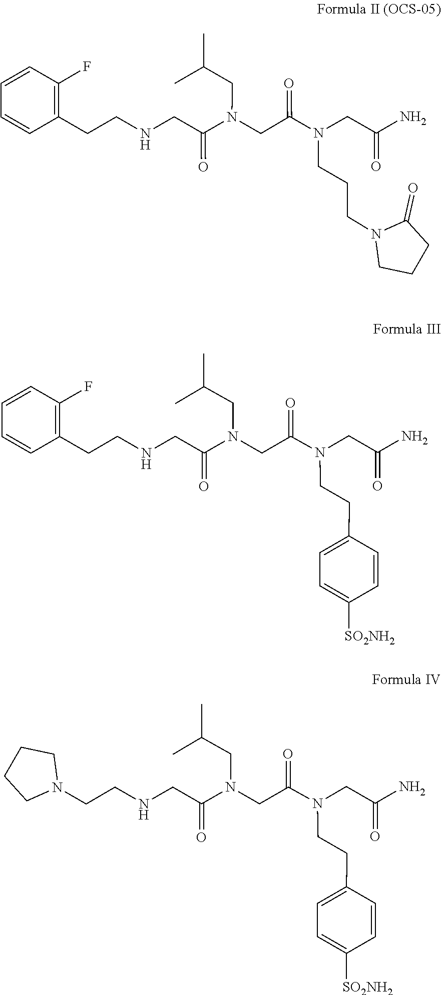 [SMILES: CC(C)CN(CC(=O)N(CCCN1CCCC1=O)CC(N)=O)C(=O)CNCCc1ccccc1F ; CC(C)CN(CC(=O)N(CCc1ccc(S(N)(=O)=O)cc1)CC(N)=O)C(=O)CNCCc1ccccc1F ; CC(C)CN(CC(=O)N(CCc1ccc(S(N)(=O)=O)cc1)CC(N)=O)C(=O)CNCCN1CCCC1]

or a pharmaceutically acceptable salt thereof.
72. The compound for use of embodiment 71, wherein the compound of Formula I is Formula II, or a pharmaceutically acceptable salt thereof.
73. The compound for use of embodiment 71, wherein the compound of Formula I is Formula III or a pharmaceutically acceptable salt thereof.
74. The compound for use of embodiment 71, wherein the compound of Formula I is Formula IV or a pharmaceutically acceptable salt thereof.
75. The compound for use of any one of embodiments 68-74, wherein the compound of Formula I is administered via an intravenous injection.
76. The compound for use of any one of 68-74, wherein the compound of Formula I is administered via a intravitreal injection.
77. The compound for use of any one of 68-74, wherein the compound is administered via subcutaneous injection.
78. The compound for use of any one of 68-74, wherein the compound is administered via a topical formulation.
79. The compound for use of any one of 68-74, wherein the compound is administered via intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, or tear duct injection, or through a mucus, mucin, or a mucosal barrier, in an immediate or controlled release fashion.
80. The compound for use of any one of 68-79, wherein the treatment is initiated within 12 days of onset of symptoms.
81. The compound for use of any one of 68-80, wherein the treatment is administered on top of standard of care.
82. The compound for use of embodiment of embodiment 81, wherein the standard of care is an anti-inflammatory steroid.
83. The compound for use of any one of 68-82, wherein the optic neuropathy is acute optic neuritis.
84 The compound for use of any one of 68-82, wherein the optic neuropathy is anterior ischemic optic neuropathy, particularly a non-arteritic anterior ischemic optic neuropathy, 85. The compound for use of any one of 68-82, wherein the optic neuropathy is caused by multiple sclerosis.
86. The compound for use of any one of 68-82, wherein the optic neuropathy is caused by a disorder selected from Leber disease, dominant optic atrophy, glaucoma, and toxic or traumatic tumor-related optic neuropathy.
87. The compound for use of any one of 68-82, wherein the optic neuropathy is caused by Neuromyelitis Optica (NMO), MOG Associated Disease (MOGAD) lupus or an inflammation-causing infection.
88. The compound for use of any one of embodiments 68-87, wherein 3 mg/kg of the compound is administered.
89. The compound for use of any one of embodiments 68-88, wherein the compound is administered for 5 days.
90. The compound for use of any one of embodiments 68-89, wherein 3 mg/kg of the compound is administered for 5 days.
91. The compound for use of one of embodiments 68-71 or 75-90, wherein the R₁ is fluorophenyl 92. The compound for use of any one of embodiments 68-71 or 75-90, wherein R₁ is 2-fluorophenyl.
93. The compound for use of any one of embodiments 68-71 or 75-90, wherein R₁ is fluorophenyl which is further substituted with one or two substituents selected from the group consisting of halogen, (C₁₋₆)alkyl, (C₁₋₆)alkoxy, and halo(C₁₋₆)alkyl.
94. The compound for use of any one of embodiments 68-71 or 75-90, wherein R₁ is fluorophenyl which is further substituted with one or two substituents selected from the group consisting of halogen, (C₁₋₄)alkyl, (C₁₋₄)alkoxy, and halo(C₁₋₄)alkyl.
95. The compound for use of any one of embodiments 68-71 or 75-90, wherein R₁ is fluorophenyl which is further substituted with one or two substituents selected from the group consisting of halogen, methyl, ethyl, propyl, isopropyl, methoxy, ethoxy, fluoromethyl, and trifluoromethyl.
96. The compound for use of any one of embodiments 68-71 or 75-90, wherein R₁ is chlorophenyl.
97. The compound for use of any one of embodiments 68-71 or 75-90, wherein R₁ is selected from 2-chlorophenyl, 3-chlorophenyl and 4-chlorophenyl.
98. The compound for use of any one of embodiments 68-71 or 75-90, wherein R₁ is 2-chlorophenyl.
99. The compound for use of any one of embodiments 68-71 or 75-90, wherein R₁ is chlorophenyl which is further substituted with one or two substituents selected from the group consisting of halogen, (C₁₋₆)alkyl, (C₁₋₆)alkoxy, and halo(C₁₋₆)alkyl
100. The compound for use of any one of embodiments 68-71 or 75-90, wherein R₁ is fluorophenyl which is further substituted with one or two substituents selected from the group consisting of halogen, (C₁₋₄)alkyl, (C₁₋₄)alkoxy, and halo(C₁₋₄)alkyl.
101. The compound for use of any one of embodiments 68-71 or 75-90, wherein R₁ is bromophenyl.
102. The compound for use of any one of embodiments 68-71 or 75-90, wherein R₁ is selected from 2-bromophenyl, 3-bromophenyl and 4-bromophenyl,
103. The compound for use of any one of embodiments 68-71 or 75-90, wherein R₁ is 2-bromophenyl.
104. The compound for use of any one of embodiments 68-71 or 75-90, wherein R₁ is bromophenyl which is further substituted with one or two substituents selected from the group consisting of halogen, (C₁₋₆)alkyl, (C₁₋₆)alkoxy, and halo(C₁₋₆)alkyl
105. The compound for use of any one of embodiments 68-71 or 75-90, wherein R₁ is bromophenyl which is further substituted with one or two substituents selected from the group consisting of halogen, (C₁₋₄)alkyl, (C₁₋₄)alkoxy, and halo(C₁₋₄)alkyl.
106. The compound for use of any one of embodiments 68-71 or 75-90, wherein R₁ is bromophenyl which is further substituted with one or two substituents selected from the group consisting of halogen, methyl, ethyl, propyl, isopropyl, methoxy, ethoxy, fluoromethyl, and trifluoromethyl.
107. The compound for use of any one of embodiments 68-71 or 75-90, wherein R₁ is iodophenyl.
108. The compound for use of any one of embodiments 68-71 or 75-90, wherein R₁ is 2-iodophenyl, 3-iodophenyl or 4-iodophenyl.
109. The compound for use of any one of embodiments 68-71 or 75-90, wherein R₁ is 2-iodophenyl.
110. The compound for use of any one of embodiments 68-71 or 75-90, wherein R₁ is iodophenyl which is substituted with one or two substituents selected from the group consisting of halogen, (C₁₋₆)alkyl, (C₁₋₆)alkoxy, and halo(C₁₋₆)alkyl.
111. The compound for use of any one of embodiments 68-71 or 75-90, wherein R₁ is iodophenyl which is further substituted with one or two substituents selected from the group consisting of halogen, (C₁₋₄)alkyl, (C₁₋₄)alkoxy, and halo(C₁₋₄)alkyl.
112. The compound for use of any one of embodiments 68-71 or 75-90, wherein R₁ is iodophenyl which is further substituted with one or two substituents selected from the group consisting of halogen, methyl, ethyl, propyl, isopropyl, methoxy, ethoxy, fluoromethyl, and trifluoromethyl.
113. The compound for use of any one of embodiments 68-71 or 75-90, wherein R₁ is trifluoromethylphenyl.
114. The compound for use of any one of embodiments 68-71 or 75-90, wherein R₁ is 2-trifluoromethylphenyl, 3-trifluoromethylphenyl or 4-trifluoromethylphenyl.
115. The compound for use of any one of embodiments 68-71 or 75-90, wherein R₁ is 2-trifluoromethylphenyl.
116. The compound for use of any one of embodiments 68-71 or 75-90, wherein R₁ is trifluoromethylphenyl which is further substituted with one or two substituents selected from the group consisting of halogen, (C₁₋₆)alkyl, (C₁₋₆)alkoxy, and halo(C₁₋₆)alkyl.
117. The compound for use of any one of embodiments 68-71 or 75-90, wherein R₁ is trifluoromethylphenyl which is further substituted with one or two substituents selected from the group consisting of halogen, (C₁₋₄)alkyl, (C₁₋₄)alkoxy, and halo(C₁₋₄)alkyl.
118. The compound for use of any one of embodiments 68-71 or 75-90, wherein R₁ is trifluoromethylphenyl which is further substituted with one or two substituents selected from the group consisting of halogen, methyl, ethyl, propyl, isopropyl, methoxy, ethoxy, fluoromethyl, and trifluoromethyl.
119. The compound for use of any one of embodiments 68-71 or 75-90, wherein R₁ is pyrrolidin-1-yl.
120. The compound for use of any one of embodiments 68-71 or 75-90, wherein R₂ is 2-oxo-pyrrolidin-1-yl-methyl.
121. The compound for use of any one of embodiments 68-71 or 75-90, wherein R₂ is sulfamoylphenyl.
122. The compound for use of any one of embodiments 68-71 or 75-90, wherein R₂ is 2-sulfamoylphenyl, 3-sulfamoylphenyl, or 4-sulfamoylphenyl.
123. The compound for use of any one of embodiments 68-71 or 75-90, wherein R₂ is 4-sulfamoylethyl.
124. The compound for use of any one of embodiments 68-71 or 75-90, wherein R₃ is 2-methylpropyl.
125. The compound for use of any one of embodiments 68-71 or 75-90, wherein R₁ is 2-fluorophenyl or pyrrolidin-1-yl, and R₂ is 2-oxo-pyrrolidin-1-ylmethyl or 4-sulfamoylphenyl.
126. The compound for use of any one of embodiments 68-125, wherein said method provides a statistically significant difference in GCIPL (ganglion cell inner plexiform layer) thickness as a surrogate of RGC (retinal ganglion cells) preservation at least month 3 from administration.
127. The compound for use of any one of embodiments 68-125, wherein said method provides a statistically significant difference in RNFL (retinal nerve fiber layer) thickness as a surrogate of axon preservation up to at least month 3 from administration.
128. The compound for use of any one of embodiments 68-125, wherein said method provides a statistically significant improvement in low contrast visual acuity (LCVA) up to at least month 3 from administration.
129. The compound for use of any one of embodiments 68-125, wherein said method results in a lower incidence of adverse events related to multiple sclerosis relapses 130. The compound for use of any one of embodiments 68-125, wherein said method results in a lower incidence of adverse events related to worsening of CNS inflammatory disorders.
131. The compound for use of any one of embodiments 68-125, wherein said method results in no difference in percent of patients with abnormal electrocardiogram (ECG) events between dosed patients and placebo patients.
132. The compound for use of embodiment 68, 69 or 70, wherein the human patient is diagnosed with an optic neuropathy that is demyelinating acute optic neuritis.
133. The compound for use of any one of embodiments 68-122, wherein the human patient does not have known neuromyelitis optica with autoantibodies against aquaporin-4 (AQP4-Abs).
134. The compound for use of embodiment 68 or 69, wherein said compound is the compound of formula II

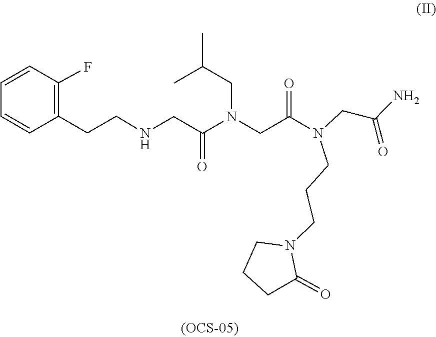 [SMILES: CC(C)CN(CC(=O)N(CCCN1CCCC1=O)CC(N)=O)C(=O)CNCCc1ccccc1F]

and said subject is having acute optic neuritis with onset of visual loss symptoms, wherein said method comprises a single treatment regimen of a once daily intravenous administration of 2-5 mg/kg for 4, 5, or 6 continuous days, said single treatment starting within the last 12 days following onset of visual loss symptoms.
135. The compound for use of embodiment 134, wherein said method comprises a single treatment regimen of a once daily intravenous administration of 3 mg/kg for 5 continuous days.
136. The compound for use of embodiment 134, wherein said visual loss symptom is a loss in Low Contrast Visual Acuity.
137. The compound for use of embodiment 134, wherein said single treatment regimen is administered in combination with an anti-inflammatory steroid treatment, wherein Low Contrast Visual Acuity at 3 months is improved, as compared to the anti-inflammatory steroid monotherapy.

### EXAMPLES

### Abbreviations Used

| | |
|---|---|
| t1, t2, | Treatment day 1, Treatment day 2, Treatment day 3, |
| t3, t4, t5 | Treatment day 4, and Treatment day 5 |
| D 15 | 15 days after conclusion of treatment |
| M 1 (D 30) | 1 month after conclusion of treatment |
| M 3 (D 90) | 3 months after conclusion of treatment |
| M 6 (D 180) | 6 months after conclusion of treatment |

### Example 1: Phase II Clinical Trial—A Randomized, Double-Blind, Placebo-Controlled Study to Evaluate the Safety and Tolerability of OCS-05 in Patients with Acute Optic Neuritis

A clinical trial was conducted investigating the use of OCS-05 to treat diagnosed demyelinating unilateral Acute Optic Neuritis (AON) in adult men and women aged 18-60 years old. In the treatment group, 3 mg/kg of OCS-05 was administered in an intravenous infusion over two hours. Subjects were treated once per day for five days. All subjects will receive corticosteroid IV as concomitant therapy unless contra-indicated, as it is the standard of care for AON.

The subjects were randomized into treatment and control groups 1:1 (eighteen subjects per group). Eligible subjects were randomized into a trial group within 12 days of onset of AON (visual loss symptoms). Randomization was stratified by visual acuity (ETDRS High contrast). Six additional subjects were included in a treatment group of OCS-05 2 mg/kg/day. Subjects were followed-up for a total of 6 months.

The trial specifically analyzed the change in Ganglion Cell and Inner Plexiform Layer (GCIPL) thickness and change in Retinal Nerve Fiber Layer (RNFL) thickness. Changes in both GCIPL and RNFL were measured in the affected eye and compared to baseline of the affected eye. The changes were measured with Optical Coherence Tomography (OCT).

Subjects were examined at day 5 of treatment (t5), one month following treatment (M1), three months following treatment (M3) and six months following treatment (M6) compared to baseline of the affected eye.

### Study Design

### Overview

A phase 2, two-armed, randomized, double-blind, study was conducted to evaluate the safety and tolerability of OCS-05, compared to placebo, in patients with acute optic neuritis (AON) receiving the standard of care. Eligible subjects aged 18 to 60, with recent onset (visual loss symptoms) of unilateral AON (idiopathic or associated with multiple sclerosis) were randomized and treated. Out of them, thirty-six (36) were randomized and treated following a 1:1 ratio to receive OCS-05 (N=18 at 3 mg/kg/Day) or placebo (N=18). Randomization was stratified by visual acuity.

Subjects in the experimental drug groups received OCS-05 IV infusion once a day for 5 days, while subjects in the placebo group received the matching placebo, an infusion of standard saline solution once a day for 5 days. All patients received corticosteroid IV as concomitant therapies unless contra-indicated, as they are standard of care of AON.

Subjects were followed-up for a total of 6 months for safety and efficacy. An interim analysis was conducted focusing on the safety endpoints when at least 20 subjects have completed the month 3 visit.

The study comprised an enrolment period, a treatment period and a follow-up period as follows.

### Enrolment: Screening (Visit 1) and Randomization (Visit 2 on Day 0)

Patients with a recent diagnosis of AON were screened at visit 1 to establish their eligibility to participate in the ACUITY study based on the inclusion and exclusion criteria. Subjects were screened after giving an informed consent. Baseline parameters were taken for future analyses. At visit 2, on day 0, eligible patients were randomized to one of the two arms (OCS-05 3 mg/kg/day or placebo). Randomization of eligible subjects should occur no later than 12 days after first AON symptoms (visual loss symptoms). Randomization was stratified by visual acuity.

### Treatment (Visit 3: T1 on Day 1 to 15)

OCS-05 administration should be initiated as early as possible. Double-blinded investigational medical product (IMP) administration (OCS-05 at 3 mg/kg or placebo) was started on day 1 (+3 days) after randomization (V2, day 0). Subjects were administered a 2-hour infusion each day, for 5 consecutive days if possible (treatment t1, t2, t3, t4 and t5). When necessary, interruptions are allowed (due to weekends, holidays and/or possible unavailability of the subject or the center), as long as the timespan for treatment does not exceed 10 days between t1 and t5. The dose was calculated based on baseline's body weight. Subjects in the control group received an infusion of normal saline solution. Blood samples were taken during the administration phase for PK analysis (secondary endpoint), biochemistry (safety endpoint) and PD analysis (exploratory endpoint).

### Follow-Up Period (Visits 4 on Day 15 to Visit 7 on D180/Month 6)

The first visit of the follow-up period (visit 4) occurred on day 15 (D15, i.e. 15 days after day 1 of treatment (t1)). Subjects were followed up for 6 months with visits at D30/month 1 (M1), D90/month 3 (M3) and D180/month 6 (M6), to evaluate the safety and preliminary efficacy of the investigational product.

### End of Study

The End of Trial was defined as either the date of the last visit of the last subject or the date of receipt of the last data point from the last subject that is required for primary, secondary and/or exploratory analysis, as prespecified in the protocol, whichever is the latest date.

### Investigational Medicinal Products

### Identity and Composition of Investigational Medicinal Products (IMP)

An overview of the study's IMPs is presented in Table 1.

TABLE 1
Overview of IMPs

| IMP | Product Name | Strength | Presentation/Form | Route |
|---|---|---|---|---|
| OCS-05 | OCS-05 solution | 500 | Lyophilised powder in vial (to be | IV |
| | for infusion | mg/vial | reconstituted with 0.9% sodium | infusion |
| | | | chloride for IV infusion) | |
| Placebo | Placebo solution | N/A | 0.9% sodium chloride sterile | IV |
| | for infusion | | saline solution (NaCl) | infusion |

### OCS-05 Description

OCS-05 is a white to off white freeze-dried powder, administered as an IV infusion after reconstitution and dilution with an appropriate volume of sterile saline solution for injection. OCS-05 is supplied in a 500 mg tube glass vial with chlorobutyl rubber stoppers and aluminum seal with cap.

Composition of OCS-05 before reconstitution is presented below in Table 2. The excipient of OCS-05, citric acid monohydrate and trisodium citrate dihydrate, are common components of buffering systems in the pH range 5 to 6.

TABLE 2
Quantitative Composition of OCS-05 Drug Product

| | Quantity | | |
|---|---|---|---|
| Component | (mg) | Function | Quality |
| OCS-05 Drug Substance | 500 | Active ingredient | In house |
| Citric acid monohydrate | 100 | Buffer agent | Ph. Eur. |
| Trisodium citrate dihydrate | 18.5 | Buffer agent | Ph. Eur. |

### Placebo

Pharmacists were not blinded to treatment. The placebo for this study was a commercial 0.9% sodium chloride sterile saline solution (NaCl) for IV infusion matching the active treatment.

### Example 2. Retinal Layers Thickness Measured by Optical Coherence Tomography (OCT)

### Retinal Nerve Fiber Layer (RNFL) Thickness

The RNFL comprises of unmyelinated axons originating from the retinal ganglion cell bodies. During AON onset, RNFL thickness first increases due to optic nerve swelling during inflammation, but post-inflammation reduction in thickness indicates axonal loss. Because RNFL is not myelinated, its thinning is direct evidence of axonal loss, as opposed to a potential demyelination and loss of oligodendrocytes. RNFL's thinning correlates with measures of persistent visual dysfunction and nerve atrophy detected by MRI.

### Ganglion Cell Inner Plexiform Layer (GCIPL) Thickness

The retinal ganglion cell layer, or retinal ganglion cell and inner plexiform layers combined (GCIPL)—the two layers are difficult to discriminate by OCT—is not affected by disk swelling during the inflammation phase of AON and is therefore more specific to neuronal damage (soma shrinkage, synapsis and axonal loss) shortly after onset of AON than RNFL thickness.

### OCT Performed by an Orthoptist or Ophthalmologist

Optical coherence tomography (OCT) acquisition was performed by an orthoptist or an ophthalmologist at the following visits: screening, t5, M1, M3, M6. The retinal thickness measurements were also performed by a central reading center.

OCT scans were used to take the below measurements according to the schedules of study procedures:
Macular thickness (μm) on both eyes (affected and fellow eye)
Thickness of the Ganglion Cell plus Inner Plexiform layer (GCIPL) (μm) on both eyes (affected and fellow eye)
Thickness of the Retinal Nerve fiber layer (RNFL) (μm) on both eyes (affected and fellow eye)

OCT parameters performed by the orthoptist or ophthalmologist were analyzed using the observed values at each visit, absolute and relative changes from baseline or from t5 to all post-baseline visits for each eye separately (affected, fellow). The algorithm of changes from baseline or from t5 is as follows:

$$Absolute Change in Macular thickness = Macular thickness at visit x - Macular thickness at baseline .$$

$$Absolute Change in RNFL thickness = RNFL thickness at visit x - RNFL thickness at baseline .$$

$$Absolute Change in GCIPL thickness at visit x - GCIPL thickness at baseline .$$

$$Relative Change in Macular thickness = 100 * ( Macular thickness at visit x - Macular thickness at baseline ) / Macular thickness at baseline .$$

$$Relative Change in RNFL thickness = 100 * ( RNFL thickness at visit x - RNFL thickness at baseline ) / RNFL thickness at baseline .$$

$$Relative Change in GCIPL thickness = 100 * ( GCIPL thickness at visit x - GCIPL thickness at baseline ) / GCIPL thickness at baseline .$$

### OCT Performed by Central Reading Center

Measurements of RNFL and GCIPL layers' thicknesses are also provided by the central Reading Center:
Global RNFL thickness for affected and fellow eye, in Cirrus™ OCT and Spectralis® OCT.
GCIPL thickness data for the 4 quadrants (Inner superior, Inner temporal, Inner inferior, Inner nasal) and the center (Central area) for affected and fellow eye, in Cirrus™ OCT and Spectralis® OCT.

OCT Cirrus™ and Spectralis® performed by the central reading center were analyzed using the observed values at each visit, absolute and relative changes from baseline or from t5 to all post-baseline visits for each eye separately (affected, fellow). The average GCIPL thickness over the 4 regions was calculated. If one region was missing, then the average GCIPL thickness was applied on the 3 remaining regions. If at least two regions were missing, then the average GCIPL thickness is missing.

The algorithm of changes from baseline or from t5 is as follows:

$$Absolute Change in RNFL thickness = RNFL thickness at visit x - RNFL thickness at baseline .$$

$$Absolute Change in GCIPL thickness ( central area , inner superior , \\ inner temporal , inner inferior , inner nasal , average of the 4 regions ) = GCIPL thickness at visit x - GCIPL thickness at baseline .$$

$$Relative Change in RNFL thickness = 100 * ( RNFL thickness at visit x - RNFL thickness at baseline ) / RNFL thickness at baseline .$$

$$Relative Change in GCIPL thickness ( central area , inner superior , \\ inner temporal , inner inferior , inner nasal , average of the 4 regions ) = 100 * ( GCIPL thickness at visit x - GCIPL thickness at baseline ) / GCIPL thickness at baseline .$$

### Categorical Variable

Absolute changes from baseline or from t5 in RNFL thicknesses assessed from orthoptist/ophthalmologist or central reading center in Cirrus™ or Spectralis® OCT was categorized into: ≤−5 μm or >−5 μm. Number (%) of subjects with a retina atrophy in each eye was tabulated. Other categories (≤−10 μm or >−10 μm; ≤−15 μm or >−15 μm) were also used.

Absolute changes from baseline or from t5 in GCIPL thicknesses assessed from central reading center, including the average of the 4 regions, in Cirrus™ or Spectralis® OCT were categorized into ≤−5 μm, >−5 μm. Number (%) of subjects with a retina atrophy at each visit in each eye separately (affected, fellow) was tabulated. Other categories (≤−10 μm or >−10 μm; ≤−15 μm or >−15 μm) were also used.

TABLE 3
GCIPL and RNFIL thickness assessed in treatment and placebo groups

| | 3 mg/kg/day | | 2 mg/kg/day | | Pooled | | Placebo + SoC | | Total | |
|---|---|---|---|---|---|---|---|---|---|---|
| | (N = 15) | | (N = 4) | | (N = 19) | | (N = 14) | | (N = 33) | |
| GCIPL thickness, | 89.3 | (8.3) | 85.9 | (17.5) | 88.6 | (10.3) | 84.3 | (13.8) | 86.8 | (11.9) |
| mean (SD) | | | | | | | | | | |
| RNFL thickness, | 104.6 | (13.1) | 174.3 | (134.1) | 119.3 | (63.1) | 115.5 | (54.1) | 117.7 | (58.6) |

mean (SD)

GCIPL mean change from baseline OCS-05 (2 or 3 mg/kg/day)+SoC compared to placebo+SoC was 8.7 μm [90% CI: 0.4; 17.1] at month 3 and 8.6 μm [90% CI: 0.2; 17.0] at month 6, reaching statistical significance (directional, nominal p value >0.05 or nominal p value >0.10) at both time points for the 2 and 3 mg/kg/day treatment groups combined.

RNFL mean change from baseline OCS-05 (3 mg/kg/day)+SoC compared to placebo+SoC was 11.1 μm [90% CI: 0.4; 21.9] at month 3 and 12.7 μm [90% CI: 1.5; 23.9] at month 6, reaching statistical significance (directional, nominal p value >0.05 or nominal p value >0.10) at both timepoints for the 3 mg/kg/day treatment group.

### Example 3. Visual Assessments

The visual function were assessed using two different charts quantifying the number of letters correctly identified by the subject:
The Early Treatment Diabetic retinopathy study (ETDRS) chart measuring high contrast visual acuity (HCVA).
The 2.5% ETDRS Low Contrast Letter Acuity chart (LCVA) measuring low contrast visual acuity.

HCVA has been the preferred historical functional outcome measure in ophthalmology, but HCVA measurement alone fails to capture important loss of visual function. Reasonable HCVA can be maintained despite poor visual function performance in other domain. LCVA better captures the persistent decrement in low contrast after AON and overcome the ceiling effect observed with HCVA measurement, where patients with close to perfect score in visual acuity struggle to function in low light situation.

In multiple sclerosis clinical trials, 2.5% low-contrast visual acuity emerged as the leading candidate to measure visual disability. Low contrast acuity testing correlated well with brain MRI lesion burden, visual evoked potentials and retinal nerve fiber layer (RNFL) loss as measured by Optical Coherence Tomography (OCT).

The visual assessments were performed at the following visits: screening, D15, M1, M3, M6 and are based on the following:
High-Contrast Visual Acuity (HCVA): HCVA is measured with the ETDRS
Low-Contrast Visual Acuity (LCVA): LCVA is measured with Sloan 2.5%

The scoring of HCVA and LCVA were based on the number of letters correctly identified. As the randomization was stratified by ETDRS HCVA (<50 letters, [50, 80 [letters, ≥80 letters). HCVA was categorized at each visit using these 3 categories.

The Snellen equivalent value was recorded in the CRF for HCVA and LCVA. The Snellen categorical response at each visit was grouped using the following categories:
worse than 20/100 (>0.7 Log MAR),
20/100 to <20/25 (0.1-0.7 Log MAR),
20/25 or better (≤0.1 Log MAR).

HCVA and Snellen data was analyzed in categories using the number (%) of subjects at each visit for each eye separately (affected, fellow).

HCVA and LCVA values were analyzed in observed values at each visit and changes from baseline to all post-baseline visits for each eye separately (affected, fellow).

The Snellen value was transformed to −log 10 (log MAR). Example: −log 10 (20/800)=1.60. The converted log MAR scores were rounded to two decimals. If the vision worsens, then the log MAR increases. A change of 0.1 on the log MAR scale represents a change of 1 line of 5 letters. If 0 letters and no light perception, then visual acuity will be set to 3.1 log MAR.

Log MAR values were analyzed using observed values at each visit and changes from baseline to all post-baseline visits for each eye separately (affected, fellow). 10 Below is the conversion table between letters, Snellen value and log MAR value (Table 4):

TABLE 4
LogMAR values

| Letter Score | LogMAR Value | Snellen Equivalent |
|---|---|---|
| 5 | 1.6 | 20/800 |
| 10 | 1.5 | 20/640 |
| 15 | 1.4 | 20/500 |
| 20 | 1.3 | 20/400 |
| 25 | 1.2 | 20/320 |
| 30 | 1.1 | 20/250 |
| 35 | 1.0 | 20/200 |
| 40 | 0.9 | 20/160 |
| 45 | 0.8 | 20/125 |
| 50 | 0.7 | 20/100 |
| 55 | 0.6 | 20/80 |
| 60 | 0.5 | 20/63 |
| 65 | 0.4 | 20/50 |
| 70 | 0.3 | 20/40 |
| 75 | 0.2 | 20/32 |
| 80 | 0.1 | 20/25 |
| 85 | 0.0 | 20/20 |
| 90 | −0.1 | 20/15 |
| 95 | −0.2 | 20/12 |

TABLE 5
Mean HVCA and LCVA measurements in treatment groups

| | 3 mg/kg/day | | 2 mg/kg/day | | Pooled | | Placebo + SoC | | Total | |
|---|---|---|---|---|---|---|---|---|---|---|
| | (N = 15) | | (N = 4) | | (N = 19) | | (N = 14) | | (N = 33) | |
| HCVA, mean | 54.1 | (34.5) | 28.5 | (28.8) | 48.7 | (34.4) | 42.6 | (34.5) | 46.1 | (34.0) |
| LCVA, mean | 19.4 | (22.3) | 19.5 | (3.0) | 15.6 | (21.1) | 17.8 | (24.3) | 16.5 | (22.2) |

TABLE 6
LCVA in Letters

| | OCS-05 + SoC | | | | Placebo + | |
|---|---|---|---|---|---|---|
| | 3 mg/kg/day | | 2 mg/kg/day | | SoC | |
| | (N=) | | (N=) | | (N=) | |
| 5 Days Post Dose | 27.42 | (7.69) | 26.93 | (13.49) | 15.36 | (7.85) |
| P value (90% CI) | 0.1408 | (0.46; 23.67) | 0.234 | (−5.06; 28.21) | — | |
| 1 Month Post | 40.26 | (5.30) | 41.46 | (11.45) | 31.12 | (5.69) |
| Treatment | | | | | | |
| P value (90% CI) | 0.12455 | (0.94; 17.33) | 0.2132 | (−3.18; 23.87) | — | |
| 3 Months Post | 48.12 | (3.93) | 44.85 | (7.77) | 29.97 | (4.22) |
| Treatment | | | | | | |
| P value (90% CI) | *0.00185 | (12.09; 24.22) | 0.053 | (5.47; 24.29) | — | |
| 6 Months Post | 51.66 | (3.76) | 45.10 | (7.45) | 36.89 | (4.03) |

| Treatment | | | | | |
|---|---|---|---|---|---|
| P value (90% CI) | *0.00595 | (8.97; 20.56) | 0.17255 | (−0.82; 17.23) | — |

LCVA mean change from baseline OCS-05 (3 mg/kg/day)+SoC compared to placebo+SoC was 18 ETDRS letters [90% CI: 8.39; 27.92] at month 3 and 15 ETDRS letters [90% CI: 5.43; 24.10] at month 6, reaching statistical significance (directional, nominal p value >0.05 or nominal p value 0.003 at month 3 and 0.015 at month 6) at both timepoints for the 3 mg/kg/day treatment group.

### Example 4: Biomarker Analyses

Analysis of neurofilament light chain (NfL) was measured. NfL are structural proteins that maintain the 3D structure of axons and are released to the extracellular space after axonal damage. From there, they are released into the blood and can be detected. Thus, plasma NfL levels are indicative of axonal damage.

Blood samples were collected for plasma NfL measurements at baseline and at day 5 and months 1, 3, and 6 and analyzed at the central laboratory using Single Molecule Array (SIMOA) technology (Quanterix Corporation, Billerica, MA) (Barro C, Leocani L, Leppert D, Comi G, Kappos L, Kuhle J. Fluid biomarker and electrophysiological outcome measures for progressive MS trials. Mult Scler. 2017 October; 23 (12): 1600-1613).

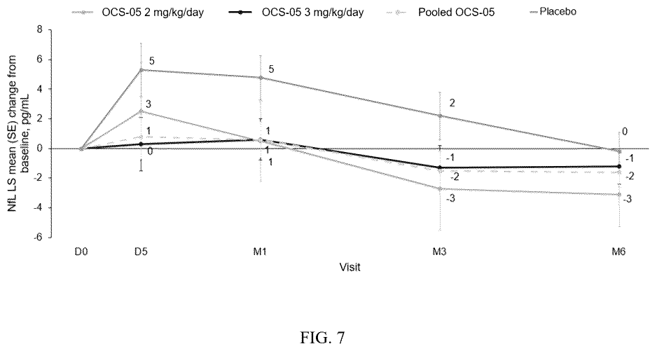

At day 5, the change from baseline in plasma NfL was lower with OCS-05 3 mg/kg/day versus placebo (LS mean [SE]: 0 [2] pg/mL vs 5 [2] pg/mL). Plasma NfL concentrations remained low up to month 6 with OCS-05, whereas the placebo group maintained the initial increase in NfL up to month 1 and then showed decline to baseline levels by month 6 (FIG. 7 and Table 7).

TABLE 7
Change From Baseline in Plasma NfL

| Difference in LS mean (SE) [90% CI] | D 5 | M 1 | M 3 | M 6 |
|---|---|---|---|---|
| Privosegtor 2 mg/kg/day vs placebo | −3 (4) [−9 to 4] | −4 (3) [−10 to 1] | −5 (3) [−10 to 1] | −3 (3) [−7 to 1] |
| Privosegtor 3 mg/kg/day vs placebo | −5 (3) [−9 to −1] | −4 (2) [−8 to −1] | −3 (2) [−7 to 0] | −1 (2) [−4 to 2] |
| Pooled privosegtor vs placebo | −5 (2) [−9 to −1] | −4 (2) [−8 to −1] | −4 (2) [−7 to 0] | −1 (2) [−4 to 1] |

D, day; LS, least-squares; M, month; NfL, neurofilament light chain.

Marked increases in NfL, a biomarker for axonal degeneration, initially found in participants who received placebo were not observed with OCS-05-treated participants, providing further support for efficacy in preventing axonal loss.

We claim:

1. A method for the treatment of optic neuropathy, comprising administering to a human in need thereof an effective amount of a compound of Formula I or a pharmaceutically acceptable salt thereof, with a single treatment regimen of a once daily administration of 2-5 mg/kg for 4, 5, or 6 days, wherein Formula I is 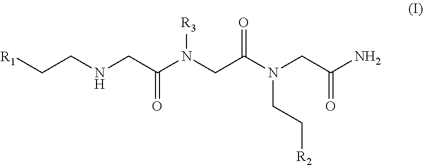
and
R₁ is phenyl substituted with halogen or trifluoromethyl, and further optionally substituted with one or two substituents selected from the group consisting of halogen, (C₁₋₆) alkyl, (C₁₋₆) alkoxy, and halo(C₁₋₆)alkyl; or
R₁ is pyrrolidin-1-yl;
R₂ is 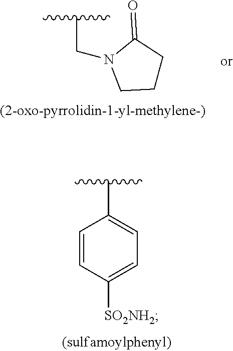 [SMILES: *CN1CCCC1=O ; *c1ccc(S(N)(=O)=O)cc1]
and
R₃ is selected from ethyl, propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl.

2. The method of claim 1, wherein the effective amount of the compound of Formula I or a pharmaceutically acceptable salt thereof, is a single regimen 4, 5, or 6 daily administrations spread over not longer than ten days.

3. The method of claim 1, wherein the effective amount of the compound administered is a single regimen of 4, 5, or 6 continuous daily administrations.

4. The method of claim 1, wherein the compound of Formula 1 is selected from 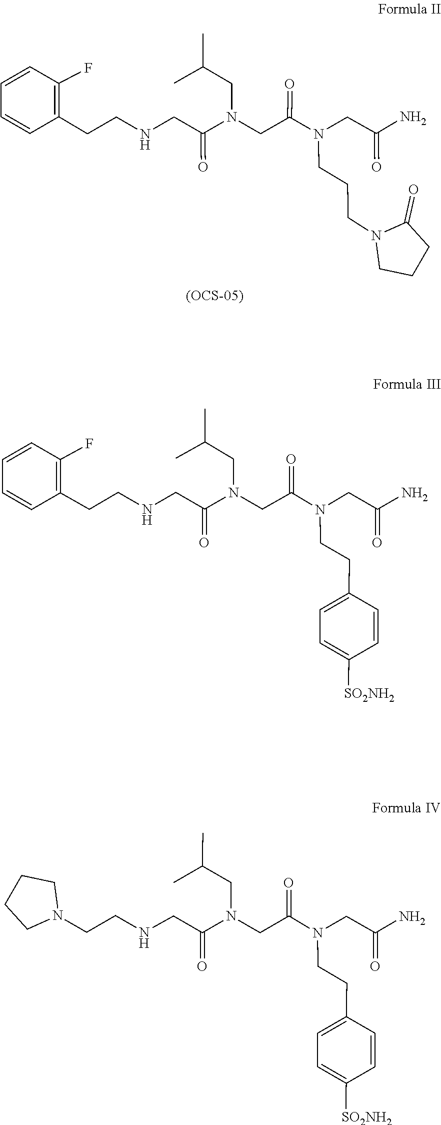 [SMILES: CC(C)CN(CC(=O)N(CCCN1CCCC1=O)CC(N)=O)C(=O)CNCCc1ccccc1F ; CC(C)CN(CC(=O)N(CCc1ccc(S(N)(=O)=O)cc1)CC(N)=O)C(=O)CNCCc1ccccc1F ; CC(C)CN(CC(=O)N(CCc1ccc(S(N)(=O)=O)cc1)CC(N)=O)C(=O)CNCCN1CCCC1]
or a pharmaceutically acceptable salt thereof.

5. The method of claim 2, wherein the compound of Formula 1 is selected from 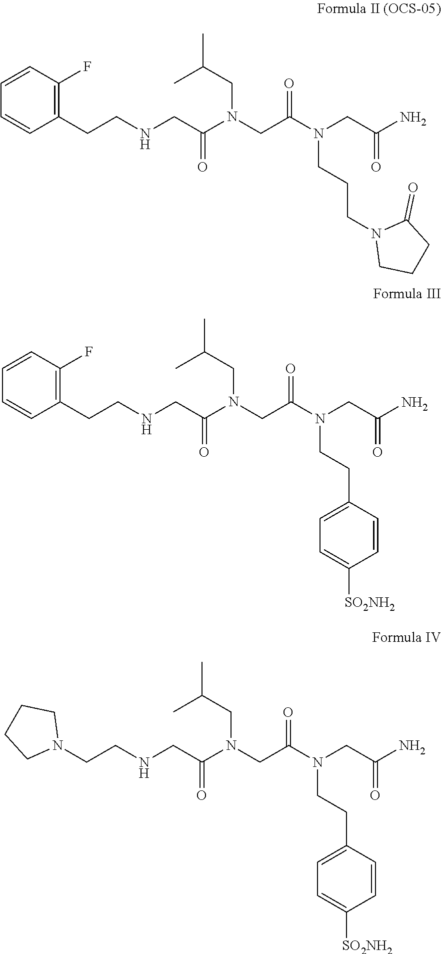 [SMILES: CC(C)CN(CC(=O)N(CCCN1CCCC1=O)CC(N)=O)C(=O)CNCCc1ccccc1F ; CC(C)CN(CC(=O)N(CCc1ccc(S(N)(=O)=O)cc1)CC(N)=O)C(=O)CNCCc1ccccc1F ; CC(C)CN(CC(=O)N(CCc1ccc(S(N)(=O)=O)cc1)CC(N)=O)C(=O)CNCCN1CCCC1]
or a pharmaceutically acceptable salt thereof.

6. The method of claim 3, wherein the compound of Formula 1 is selected from 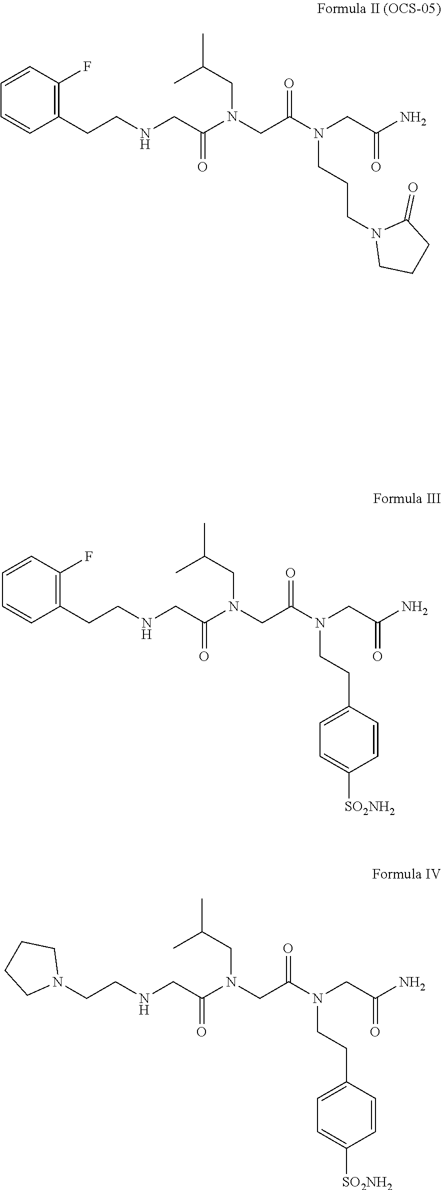 [SMILES: CC(C)CN(CC(=O)N(CCCN1CCCC1=O)CC(N)=O)C(=O)CNCCc1ccccc1F ; CC(C)CN(CC(=O)N(CCc1ccc(S(N)(=O)=O)cc1)CC(N)=O)C(=O)CNCCc1ccccc1F ; CC(C)CN(CC(=O)N(CCc1ccc(S(N)(=O)=O)cc1)CC(N)=O)C(=O)CNCCN1CCCC1]
or a pharmaceutically acceptable salt thereof.

7. The method of claim 1, wherein the compound of Formula I is administered via an intravenous injection.

8. The method of claim 1, wherein the compound of Formula I is administered via an intravitreal injection.

9. The method of claim 1, wherein the compound of Formula I is administered via subcutaneous injection.

10. The method of claim 1, wherein the treatment is initiated within 12 days of onset of symptoms.

11. The method of claim 1, wherein the treatment is administered on top of standard of care.

12. The method of claim 11, wherein the standard of care is an anti-inflammatory steroid.

13. The method of claim 1, wherein the optic neuropathy is acute optic neuritis.

14. The method of claim 1, wherein the optic neuropathy is anterior ischemic optic neuropathy

15. The method of claim 14, wherein the anterior ischemic optic neuropathy Non-arteritic Anterior Ischemic Optic Neuropathy.

16. The method of claim 1, wherein the optic neuropathy is caused by multiple sclerosis.

17. The method of claim 1, wherein the optic neuropathy is caused by Neuromyelitis Optica (NMO), MOG Associated Disease (MOGAD), lupus or an inflammation-causing infection.

18. The method of claim 1, wherein 3 mg/kg of the compound of Formula I is administered.

19. The method of claim 1, wherein the single treatment regimen provides a statistically significant difference in GCIPL (ganglion cell inner plexiform layer) thickness as a surrogate of RGC (retinal ganglion cells) preservation at least month 3 from administration.

20. The method of claim 1, wherein the single treatment regimen provides a statistically significant difference in RNFL (retinal nerve fiber layer) thickness as a surrogate of axon preservation up to at least 3 months from administration.

21. The method of claim 1, wherein the single treatment regimen provides a statistically significant improvement in low contrast visual acuity (LCVA) up to at least 3 months from administration.

22. The method of claim 1, wherein the single treatment regimen results in a lower incidence of adverse events related to multiple sclerosis relapses.

23. The method of claim 1, wherein the compound of Formula I is

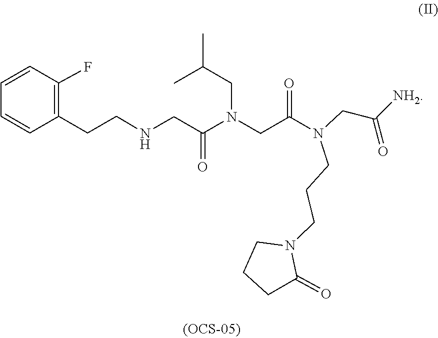 [SMILES: CC(C)CN(CC(=O)N(CCCN1CCCC1=O)CC(N)=O)C(=O)CNCCc1ccccc1F]

24. The method of claim 2, wherein the compound of Formula I is

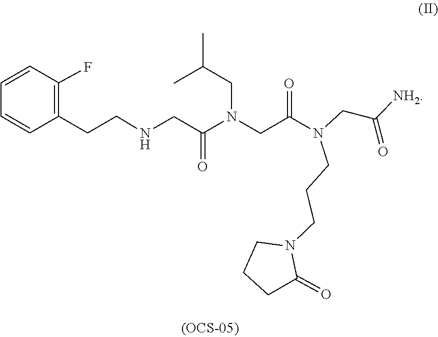 [SMILES: CC(C)CN(CC(=O)N(CCCN1CCCC1=O)CC(N)=O)C(=O)CNCCc1ccccc1F]

25. The method of claim 3, wherein the compound of Formula I is 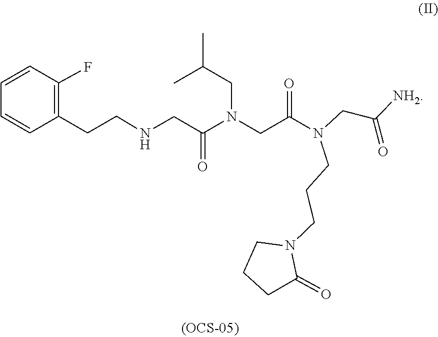 [SMILES: CC(C)CN(CC(=O)N(CCCN1CCCC1=O)CC(N)=O)C(=O)CNCCc1ccccc1F]
